(19) United States
(12) Patent Application Publication
(10) Pub. No.: US 2024/0090814 A1
(43) Pub. Date: Mar. 21, 2024

(54) RAPID MANUFACTURING OF ABSORBENT SUBSTRATES FOR SOFT, CONFORMABLE SENSORS AND CONDUCTORS

(71) Applicant: THE TRUSTEES OF THE UNIVERSITY OF PENNSYLVANIA, Philadelphia, PA (US)

(72) Inventors: Flavia VITALE, III, Wilmington, DE (US); Nicolette DRISCOLL, Somerville, MA (US); Nicholas V. APOLLO, San Diego, CA (US); Brian LITT, Bala Cynwyd, PA (US)

(21) Appl. No.: 17/767,709

(22) PCT Filed: Oct. 9, 2020
(86) PCT No.: PCT/US2020/055147

§ 371 Date: Apr. 8, 2022

Related U.S. Application Data
(60) Provisional application No. 62/913,824, filed on Oct. 11, 2019.

(51) Int. Cl. A61B 5/27; A61B 5/00; A61B 5/268

(52) U.S. Cl. CPC A61B 5/27; A61B 5/268; A61B 5/6846; A61B 2562/125; A61B 2562/164

(57) ABSTRACT

Provided are are conformable conductors and electrode arrays and related methods of their manufacture and use. The disclosed structures can be implanted into or placed outside of the body of a subject to record biosignals and/or to deliver electrical stimulation, in addition to other, non-biological applications for electrical and/or chemical sensing and stimulation. One can form a pattern an absorbent material (e.g., with a laser cutter), which is later infused with a conductive ink that can include, e.g., MXene materials, reduced graphene oxide (rGO), graphene/graphite, gold, platinum, or other metallic nanoparticles, carbon nanotubes, conductive polymers, or other conductive ink materials. The resulting electrode arrays can be compatible with magnetic resonance imaging (MRI or fMRI) and transcranial magnetic stimulation (TMS) modalities, and the disclosed process can rapidly produce electrodes at high yield.

### RELATED APPLICATIONS

The present application claims priority to and the benefit of U.S. patent application No. 62/913,824, “Rapid Manufacturing of Absorbent Substrates For Soft, Conformable Sensors” (filed Oct. 11, 2019), the entirety of which foregoing application is incorporated herein by reference for any and all purposes.

### GOVERNMENT RIGHTS

This invention was made with government support under Contract No. DGE-1845298 awarded by the NSF. The government has certain rights in the invention.

### TECHNICAL FIELD

The present disclosure relates to the field of flexible electrodes and to the field of conductive textiles.

### BACKGROUND

The ability to acquire high-fidelity biosignals from excitable tissues is paramount to clinical diagnostics, research, and emerging closed-loop therapeutics. At present, electrode arrays are utilized with varying invasiveness, ranging from external electroencephalogram (EEG) and electromyography (EMG) recordings on the skin surface, to intracortical recordings in which electrodes are implanted on the brain's surface or into brain tissue.

Electrode arrays that are commercially available are made almost exclusively from noble metals and can be rigid and difficult to use comfortably with patients. Further, the fabrication process of such arrays is tedious and requires considerable labor and resources. In addition, existing devices are expensive and generally only used once in a clinical setting, as they are disposed of following human use. Accordingly, there is a long-felt need in the field for improved flexible electrodes and related methods of manufacturing such materials.

### SUMMARY

In meeting the described long-felt needs, the present disclosure first provides components, comprising: (a) one or more sensors, a sensor comprising: (i) a permeable substrate material having an upper surface, the permeable substrate optionally being non-conductive; and (ii) an electrically conductive material, the electrically conductive material disposed and/or in within the permeable substrate material so as render the permeable substrate material conductive; and (b) an insulating material, the insulating material having an upper surface and a thickness, and the insulating material defining at least one aperture extending through the thickness of the insulating material, the at least one aperture being in register with a sensing location on the upper surface of the permeable substrate material of a sensor.

Also provided are methods, comprising: collecting a signal with a component according to the present disclosure.

Additionally provided are methods, comprising: fabricating a component according to the present disclosure.

Further disclosed are devices, the devices comprising: one or more components according to the present disclosure.

Also provided are methods, comprising: infusing a fluid that comprises a carrier and a conductive material into a permeable substrate portion and then removing at least some of the carrier, the infusing and removing being carried out under such conditions that the conductive material renders the permeable substrate conductive; disposing an electrically insulating material over the permeable substrate, the electrically insulating material having an upper surface and defining a thickness; optionally disposing a sealant over the electrically insulating material; forming an opening through the thickness of the electrically insulating material, the opening being in register with a sensing location on the permeable substrate.

Additionally provided are components, comprising: (a) one or more sensors, a sensor comprising: a conductive permeable substrate material having an upper surface; and (b) an insulating material, the insulating material having an upper surface and a thickness, and the insulating material defining at least one aperture extending through the thickness of the insulating material, the at least one aperture being in register with a sensing location on the upper surface of the permeable substrate material of a sensor.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIG. 1. Fabrication schematic. (A) laser patterning of textile substrate (light blue) placed atop glass slide. (B) Injection of conductive ink (purple) to fill trace. Surrounding textile not utilized in sensor can be removed. (C) Overmolding with PDMS (blue) or other encapsulant rubber and (D) removal of insulation material at electrode face using biopsy punch or laser cutter.

FIG. 2. Experimental setup for EMG measurement. MXene-PDMS array was fixed to the forearm, the ground electrode was placed on the inner wrist, and the reference electrode was placed on the elbow. The subject held a load cell between thumb and forefinger, and applied cyclic loading to the load cell to activate the muscles of the forearm.

FIG. 3. Early prototypes of the MXene-textile-PDMS electrode array (MXene-containing electrodes can also be termed MXtrodes, which term is used herein for convenience in some instances). (A) Electrode array following laser patterning and MXene injection. The laser burns diffusion barriers between channels so that MXene only fills one channel at a time. (B) Completed device following PDMS encapsulation and opening of the electrode contact.

FIG. 4. (A) DC Resistance of MXene and PEDOT:PSS textile wires. Note that the DC resistance is linear with length, however data are displayed here on a log scale for better visualization. (B) Resistance from electrode to contact for 2 generations of the MXene-textile-PDMS device. MT03 had 1 mm wide interconnects while MT04 had 2 mm wide interconnects.

FIG. 5. CV of two sample MXene electrodes from two separate devices: MT04-1 (A,B) and MT04-2 (C,D). While the electrode in (A) appears to corrode as expected, (D) appears to exhibit significantly less current loss.

FIG. 6. Depiction of anodic and cathodic charge regions for 2 devices: (A) MT04-1 and (B) MT04-2.

FIG. 7. EIS data for all 8 channels on MXene-textile devices MT04-1 and MT04-2. (A) The average impedance magnitude as a function of frequency and the average phase shift (B). The error bars represent standard deviation. The inset in (B) is the equivalent circuit model used for fitting, modified from the ‘coated electrode model’ available from Gamry's model database.

FIG. 8. EIS data comparing MXene against PEDOT:PSS as the conductive ink used for the textile device. MXene-textile electrodes (such electrodes can be termed “MXtrodes,” as described elsewhere herein) show nearly an order of magnitude decrease in electrode impedance compared to PEDOT-textile electrodes.

FIG. 9. EMG recordings from two MXene-textile electrode arrays: MT04-1 (left) and MT04-2 (right). Recordings from 3 representative channels for each device are shown in black, however each device had 8 functional channels which recorded EMG signal, though there was channel-to-channel variability in terms of impedance and background noise. The force applied by the subject to the load cell is shown in blue. The subject applied a range of forces to the load cell, and the magnitude of the EMG response corresponded to the amount of force applied to the load cell, as expected.

FIGS. 10A-B. Protruding electrodes utilizing absorbent cellulose sponges. (A) Two images of a textile device, with added cellulose sponge. Following injection of the conductive ink (MXene shown here) the cellulose sponges expand upward. After drying, these form rigid pillar-like conductive structures. (B) Light microscopy images of several protruding electrodes in a finished device, showing their 3-dimensional structure. After encapsulation in PDMS, the pillars were trimmed to the desired height.

FIGS. 11a-11e provide example methods. FIG. 11a—Schematic of the fabrication method for laser-patterned planar and 3D pillar MXene electrode arrays. FIGS. 11b-11e—Photographs of different electrode array geometries (top) with their intended bioelectronic applications (bottom) for FIG. 11b—EMG, FIG. 11c—ECG, FIG. 11d—EEG, and FIG. 11e—ECoG monitoring. Scale bars: FIGS. 11b-11d 5 mm; inset in FIG. 11d and FIG. 11 e 2 mm.

FIGS. 12a-12d provide example EIS data. FIG. 12a—EIS spectra measured in 1× PBS for 3 mm, 2 mm, 1 mm, and 500 μm planar MXene electrodes compared to 2.3 mm Pt electrodes. FIG. 12b—CVs for 3 mm planar MXtrode and 2.3 mm Pt electrodes scanned from −0.6-+0.6 V at 50 mV s⁻¹. FIG. 12c—Voltage transients in response to biphasic current pulses, with t_{c}=tₐ=500 μs and tᵢₚ=250 μs, current amplitudes ranging from 1 to 5 mA for 3 mm planar MXtrode and 2.3 mm Pt electrodes. Anodic and cathodic voltage limits for MXene and Pt are displayed on their respective plots as dashed red lines. FIG. 12d—EIS spectra measured on skin for 3 mm MXtrode 3D pillar and planar electrodes.

FIGS. 13a-13f provide electrode images. FIG. 13a—Images of a MXtrode 3D EEG array with eight 3 mm-diameter MXene electrodes in a circular arrangement around a central opening. FIG. 13b—Image of MXtrode electrode array and standard gelled Ag/AgCl cup electrode placed on head of human subject. FIG. 13c—Map of 1 kHz impedance values for all electrodes on the subject's head. FIG. 13d—Segments of recorded EEG signal from all electrodes during the eyes open (left) and eyes closed (right) tasks at resting state. FIG. 13e—Spectrograms of the EEG signal recorded on MXene electrode b in the eyes open (top) and eyes closed (bottom) conditions. Alpha frequency band is enclosed in dashed box to highlight differences between eyes open and eyes closed states. FIG. 13f—Power spectral density during eyes open and eyes closed EEG recordings. The 8-12 Hz alpha band is highlighted.

FIGS. 14a-14d provide EMG recordings from the APB muscle, with: FIG. 14a—Average evoked response following N=10 median nerve stimulation epochs recorded on 20-ch MXtrode array placed over APB. Blue ticks indicate time of nerve stimulation, and purple dots indicate time of peak evoked response. FIG. 14b—Latency map of peak response overlaid on photo of the MXtrode array on the APB. White “x” indicates the channel with shortest latency, corresponding to the IZ. c-d EMG recordings from the biceps muscle. FIG. 14c—Average evoked response following N=10 supraclavicular nerve stimulation epochs recorded on 40-ch MXtrode array placed over the biceps. Blue ticks indicate time of nerve stimulation, and purple dots indicate time of peak evoked response. FIG. 14d—Latency map of peak response overlaid on photo of the MXtrode array on the subject's biceps. Distributed IZ running perpendicular to the muscle is apparent as the band with the shortest latency.

FIGS. 15a-15c provides electrocardiography with MXtrodes. FIG. 15a—Photo of ECG recording setup on human subject. Electrodes were interchanged in the same locations to obtain sequential recordings from either dry MXene or pre-gelled commercial electrodes. FIG. 15b—Ten seconds of ECG recordings on the dry MXtrodes (top) and the pre-gelled commercial electrodes (bottom). FIG. 15c—Average ECG waveforms recorded on the two electrode types, marked with salient ECG features.

FIGS. 16a-16e provides ECoG recording with MXtrode arrays in swine brain. FIG. 16a—Schematic depicting ECoG recording setup with the 6-ch array of 500 μm-diameter MXtrodes placed subdurally on somatosensory cortex. FIG. 16b—A few seconds of representative ECoG data recorded on the MXtrode array. FIG. 16c—Power spectral density of the ECoG recording, illustrating the low-noise quality of the ECoG signals obtained, evidenced by the lack of a 60 Hz noise peak. FIG. 16d—Segment of ECoG data, displayed according to the spatial arrangement of the 6 MXtrodes. FIG. 16e —Instantaneous snapshots of the voltage mapping recorded across the 6 MXtrodes reveals stereotyped patterns during down states (panels 1 and 4) and up states (panels 2 and 3). The timing of these voltage snapshots is indicated in FIG. 16e by the vertical lines. Voltage was interpolated across the array and normalized, with black dots indicating location of the 6 MXtrode contacts.

FIGS. 17a-17d provide cortical stimulation with MXtrode arrays in rat brain. FIG. 17a—Schematic of the cortical stimulation setup, with the 4-ch array of 500 μm-diameter MXtrodes placed over barrel cortex, and the optical micrometer used to detect and amplify the whisker deflection signal. FIG. 17b—Whisker deflection data recorded by the optical micrometer during a series of stimulation pulse trains delivered at 1.4 mA. FIG. 17c—Average first whisker deflection for each stimulation pulse amplitude, time-aligned by the stimulation onset. FIG. 17d—Whisker deflection amplitude scales with stimulation amplitude, with stimulation at 1.0 mA falling below the threshold required to evoke whisker movement.

FIGS. 18a-18c provides MRI and CT compatibility of MXtrodes. FIG. 18a—Schematic of the device phantom used for MRI and CT imaging. Strips of disk electrodes (3 mm-diameter MXtrodes or 2.3 mm-diameter clinical Pt ECoG strip electrodes) were embedded in a conductive agarose phantom in a glass test tube and images were taken of the cross-section. FIG. 18b—A high-field 9.4T MRI scan showing significant shadow artifact and image distortion around the Pt electrode (top), but no visible artifact from MXtrodes (bottom). The MXene contact is indistinguishable from the PDMS encapsulation in the device. FIG. 18c—High-resolution CT scans with Pt electrodes (top) showing significant x-ray scattering artifacts, while no artifact is visible from the MXtrodes (bottom).

FIGS. 19a-19e provide optical and SEM images of MXtrode composites. Optical microscopy images (top panel) and corresponding SEM images (bottom panel) for: FIG. 19a—pristine cellulose/polyester blend substrate, FIG. 19b—the same substrate after infusing with MXene ink, FIG. 19c—cross-section of MXene composite trace embedded in PDMS, FIG. 19d—edge of planar electrode contact, e side of MXene-infused cellulose foam in 3D mini-pillar MXtrode.

FIGS. 20a-20c provide Scalable fabrication of MXtrode arrays. FIG. 20a—Photo of laser-patterned array substrates for various device and array geometries. FIG. 20b—the same batch of devices shown in a after infiltrating with MXene ink. In the top left are shown the EEG ring MXtrode arrays after addition of the 3D pillars. FIG. 20c—Photographs of completed devices (from left to right) designed for ECG, ECoG, EMG, and EEG sensing.

FIGS. 21a-21e provide DC conductivity of ink-infused composites. FIG. 21a—Plot of length vs. DC resistance for composites made using MXene, PEDOT:PSS, and rGO as the conductive ink. Test structures were 20 cm×3 mm×285 μm (L×W×H) strips. FIGS. 21b-21d—Individual plots of DC resistance vs. length for b MXene, c PEDOT:PSS, and d rGO, with linear fitting curves shown as dashed lines. The linear relation of resistance vs. length, along with the cross-sectional area of the test structure, is used to compute the bulk conductivity, σ, of each of the composite materials. DC resistance of the rGO composite could only be measured out to 8 cm due to high resistance. FIG. 21e—Demonstration of exceptional conductivity of the MXene composite here used as conductive trace to power an LED.

FIGS. 22a-22k provide a scaling of electrochemical properties for MXtrodes. FIG. 22a—CVs probing the negative voltage limit for MXene. Hydrolysis of water begins at −1.9 V. FIG. 22b—CVs probing the positive voltage limit for MXene. Current loss and loss of capacitive behavior begins at +0.7 V. Test electrode for a and b was 3 mm-diameter planar MXene. FIG. 22c—CVs in MXene voltage window, −1.8-+0.6 V, for 3 mm, 2 mm, 1 mm, and 500 μm-diameter MXtrodes. FIG. 22d—CVs in MXene-Pt intersection window, −0.6-+0.6 V, for 3 mm, 2 mm, 1 mm, and 500 μm-diameter planar MXtrodes and 2.3 mm-diameter Pt electrode. FIG. 22e—Charge storage capacity of scaled planar MXtrodes as a function of diameter, highlighting the CSC scaling dependence on the electrode diameter, due to edge effects. CSC values were calculated for CVs in MXene water window. FIGS. 22f-22i—Voltage transients for biphasic current pulses, with t_{c}=tₐ=500 μs and tᵢₚ=250 μs, for currents ranging from 1 to 5 mA for f 3 mm, g 2 mm, h 1 mm, and i 500 μm-diameter planar MXene electrodes. FIG. 22j—Charge injection capacity of scaled planar MXtrodes as a function of diameter, highlighting the CIC scaling dependence on the electrode diameter, due to edge effects. FIG. 22k—Schematic demonstrating the relative electrode sizes used in the study.

FIGS. 23a-23b provide EEG alpha bandpower mapping, with FIGS. 23a—8-12 Hz alpha bandpower across the 2 min recording in the eyes open state. Color plot (top) shows average alpha value for each electrode, mapped to its corresponding location on the scalp. Bar plot (bottom) shows average alpha values, with error bars corresponding to S.E.M. of alpha values across all time windows. No significant difference was detected between the gelled Ag/AgC1 electrode (labelled i) and the dry MXtrode electrodes (labelled a-h). FIG. 23b—the same alpha band power analysis shown in FIG. 23a, for the eyes closed task.

FIGS. 24a-24b provide motor EEG recording. FIG. 24a—Photograph of the EEG recording setup with electrodes centered over the hand motor region, as localized using single TMS pulses. FIG. 24b—PSDs of the recorded EEG signal reveal a suppression of the 8-12 Hz motor mu rhythm during actual hand flexion as compared to imagined hand flexion.

FIGS. 25a-25d provide additional data from EMG experiments. FIGS. 25a-27b provide 1 kHz impedance magnitude maps for the (FIG. 28a) 20-ch planar MXtrode array used to map the APB muscle and the (FIG. 25b) 40-ch planar MXtrode array used to map the biceps, overlaid on images of the arrays on the subject during the experiment. FIG. 25c—Schematic showing the arrangement for bipolar signal subtraction shown in (FIG. 25d) for resisted flexion EMG recordings on the biceps. The latency map obtained from the supraclavicular stimulation experiment is shown overlaid on the bottom image. FIG. 25d—Bipolar EMG signals recorded during resisted flexion on the biceps. The location of the innervation zone is indicated by the arrows, and is clear from the propagation of the EMG signal outward from this region with some delay, as well as the inversion of the spikes on either side. The innervation zone determined from this analysis is in agreement with the region identified by the electrical stimulation-derived latency map shown in (FIG. 25c).

FIGS. 26a-26d provide electrooculography with MXtrodes. FIG. 26a—Schematic of EOG recording for monitoring up-down eye movements. FIG. 26b—EOG data recorded on MXtrodes, showing distinct up and down eye movements. FIG. 26c—Schematic of EOG recording for monitoring left-right eye movements. FIG. 26d—EOG data recorded on MXtrodes, showing distinct left and right eye movements.

FIGS. 27a-27b provide long-term skin impedance stability of MXtrodes. FIGS. 27a-27b provide area-normalized impedance at (FIG. 27a) 1 kHz and (FIG. 27b) 10 Hz for 3 mm-diameter planar MXtrodes and 10 mm-diameter pre-gelled commercial disk electrodes in contact with human skin for 54 hrs.

FIGS. 28a-28c provide 3T MRI compatibility and magnetic susceptibility of MXene. FIG. 28a—MXtrode 3D pillar EEG array, imaged in a 3T clinical MRI with a T2 weighted sequence. The array was placed atop an MRI phantom and a Vitamin E marker was placed on top of the MXtrode array. The Vitamin E marker is visible, while the MXtrode array is not. FIG. 28b—Thermal IR image of MXtrode EEG array captured immediately after a 10 min MRI sequence, showing no sign of heating. Left image shows MXtrode array atop the MRI phantom, and right image shows the thermal image overlay. FIG. 28c—Magnetic susceptibility of Ti₃C₂ MXene measured at body temperature, 310 K, for applied magnetic field up to 9T. For calculation of the magnetic susceptibility value, X, magnetization and field strength were both converted to units of A/m such that X is unitless.

FIG. 29 provides an exemplary cutaway view of exemplary devices according to the present disclosure.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable, and it should be understood that steps can be performed in any order.

It is to be appreciated that certain features of the invention which are, for clarity, described herein in the context of separate embodiments, can also be provided in combination in a single embodiment. Conversely, various features of the invention that are, for brevity, described in the context of a single embodiment, can also be provided separately or in any subcombination. All documents cited herein are incorporated herein in their entireties for any and all purposes.

Further, reference to values stated in ranges include each and every value within that range. In addition, the term “comprising” should be understood as having its standard, open-ended meaning, but also as encompassing “consisting” as well. For example, a device that comprises Part A and Part B can include parts in addition to Part A and Part B, but can also be formed only from Part A and Part B.

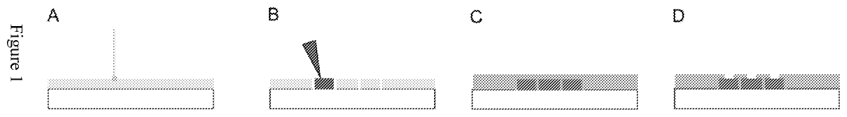

The present disclosure provides, inter alia, low-cost, environmentally-friendly sensors by using novel materials and developing a scalable manufacturing procedure. Such devices can be produced by creating circuit patterns in absorbent substrates and then infusing conductive ink into the patterns to create conductive structures (FIG. 1). The disclosed manufacturing techniques can be applied in a batch manner, but can also be applied in a roll-to-roll manner so as to effect continuous manufacturing.

In one exemplary embodiment, a cellulose/polyester textile was used as the absorbent material, two water-based conductive inks were demonstrated: Ti₃C₂ MXene and commercially available PEDOT:PSS. PDMS was used as the encapsulation material. This method is compatible with a number of alternative absorbent substrates, conductive inks, and encapsulation materials, including biodegradable rubbers such as EcoFlex™.

To achieve high electrical conductivity through the interconnects and low interfacial impedance, a conductive MXene ink was chosen to produce sensing devices prototypes. MXenes are a family of two-dimensional carbides and nitrides such as Ti₂C, Mo₂C, Ti₃C₂, etc. which are of particular interest for use in this fabrication process due to their high conductivity, biocompatibility, and inherent hydrophilicity which enables the production of stable colloidal solutions of MXene in water without the need for surfactants or strong acids. Although Ti₃C₂ MXene is used in exemplary embodiments herein, it should be understood that such embodiments are illustrative only and that conductive materials besides Ti₃C₂ MXene can be used, e.g., graphite, graphene, and other MXenes besides Ti₃C₂ MXene.

### Methods

A variety of example methods and components are described below. It should be understood that these are exemplary only and do not limit the scope of the present disclosure or the appended claims. As an illustration, although certain examples herein utilize MXene materials as conductive materials, these examples are illustrative only, as other conductive materials (besides MXenes) can be used in the disclosed technology.

### Fabrication of Electrode Structures

Absorbent pads comprised of 55% cellulose/45% polyester (Technicloth; TX609) were patterned using a CO₂ laser. For MXene devices, solutions of Ti₃C₂ MXene were produced using previously established methods and concentrations of 12 mg/mL were injected into the absorbent material to create the interconnects, electrodes, and contact pads instantly via capillary action of the absorbent material. For PEDOT:PSS devices, a high conductivity grade 1.1% PEDOT:PSS dispersion in water (Sigma Aldrich) was injected into the absorbent material using the same procedure. Following injection of the chosen conductive ink into the absorbent material, the composite was dried on a hot plate at 125° C. for 20 minutes to dry, which can improve electrical conductivity, though it is not a requirement.

### PDMS/Parylene-C Encapsulation

PDMS in a 1:10 (cure:base) ratio was poured directly onto the conductive ink-infused textile, degassed under vacuum for 20 min, and cured. During vacuum exposure, PDMS penetrates into the microstructure of the textile, giving it strength and flexibility. After PDMS curing, devices can optionally be encapsulated in Parylene-C to provide an additional barrier against moisture uptake. This step can be useful (but is not required) for implantable devices; this step is not necessary for most skin-based biosensing applications, especially if the device is used as a consumable as is common practice in clinical monitoring or diagnostic procedures.

### Exposing Electrode Site

Electrode openings were created by cutting through the top layers of PDMS and/or Parylene-C using a 3 mm biopsy punch, then removing the disks of insulating material and exposing the conductive ink-infused textile. This step can also be achieved by, e.g., a laser cutting process, which can further speed up the fabrication and reduce manual steps.

An additional step (not shown here) is the spray-coating of additional conductive material at the final step. This can effectively bring the contact area of the electrode above the insulation, i.e., such that the “top” of the electrode is not flush with the upper surface of the insulation. An enzyme, electrocatalytic element, or even a biomolecule can be included so as to enable biosensing.

### Electrochemical Testing

Electrochemical impedance spectroscopy (EIS) and cyclic voltammetry (CV) were utilized to study the charge transfer properties of these electrodes. A standard 3-cell set-up was used in which 1 of the 8 channels of device served as the working electrode, AglAgC1 was used as the reference electrode, and a graphite rod was used as the return electrode. The electrolyte was 50 mL of 10 mM phosphate buffered saline (PBS).

### In vivo Recording of EMG Signal

Electromyography (EMG) signals were measured by placing MXene-PDMS electrode arrays onto the forearm of a human subject in a ‘dry’ configuration (e.g., no conductive gel). The skin was prepared using 3M RedDot skin prep tape and wiping with 10 mM PBS. Natus EMG adhesive electrodes were used for the ground (placed on the inner wrist) and reference (placed on the elbow) electrodes. The electrode configuration is shown in FIG. 2. Signals were recorded using an Intan RHS2000 Stimulation/Recording amplifier. The subject pinched a load cell (2 kg) between thumb and forefinger in a “cyclic loading” fashion in order to activate the muscles of the forearm to various extents.

### Exemplary Results

A variety of non-limiting combinations of starting materials were evaluated. These combinations were as follows:
(i) cellulose-polyester textile/MXene ink/PDMS
(ii) cellulose-polyester textile/PEDOT:PSS ink/PDMS
iii) cellulose-polyester textile/MXene ink/PDMS+parylene-C
(iv) cellulose-polyester textile/MXene ink/EcoFlex™
(v) 100% cellulose textile/MXene ink/EcoFlex™

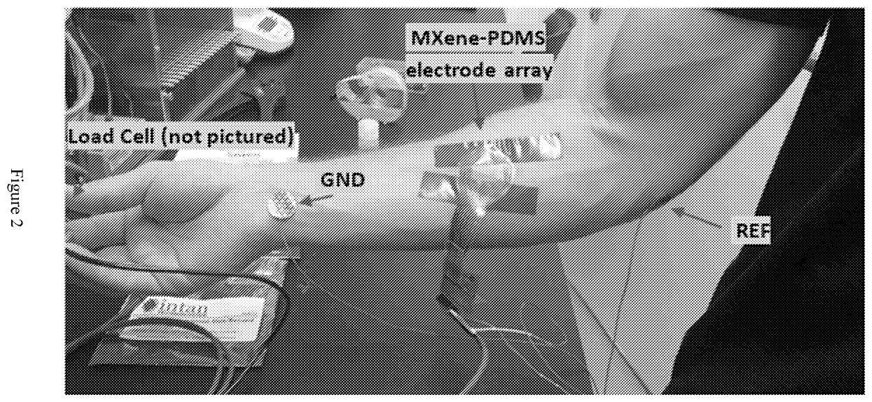

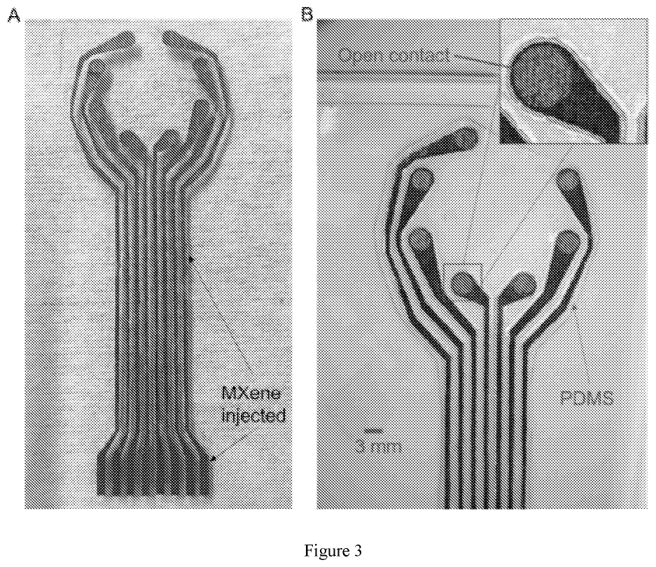

An embodiment on PDMS, cellulose-polyester textile, and MXene conductive ink is provided in FIG. 3.

The DC conductivity of the composite conductive material was characterized using two methods. First, strips of cellulose-polyester textile-conductive ink-PDMS composite material were made into 2-mm wide wires and their resistance was characterized as a function of length. This was done for composites using both MXene and PEDOT:PSS as the conductive ink for comparison. From curve fitting, the nominal resistance per length of 2 mm wide textile wires is approximately 42.75 Ω·cm-1 for MXene-textile wires and 8346.79 Ω·cm⁻¹ for PEDOT:PSS-textile wires. Thus, the resistance per length of the PEDOT:PSS wires was ˜195 times larger than that for the MXene wires, indicating that MXene has superior electrical conductivity (FIG. 4A). Additionally, DC resistance of the MXene-textile wires did not increase after encapsulation in PDMS, indicating that PDMS encapsulation does not interfere with the conductivity of the conductive ink-infused textile. For completed MXene electrode arrays, a handheld multimeter was used to measure line resistance between electrode contact and connector end contact prior to electrochemical characterization (FIG. 4B). Dependence of resistance on wire length can be a consideration in the production of “all-conductive-textile” devices which do not exhibit the same order of conductivity as metals. The present disclosure thus allows for maximization of conductivity without loss of mechanical strength or flexibility. For example, conductive rubber composites often lose flexibility as the conductivity increases due to the loading of large amounts of conductive material into the polymer matrix, which shortcoming is avoided in the present technology.

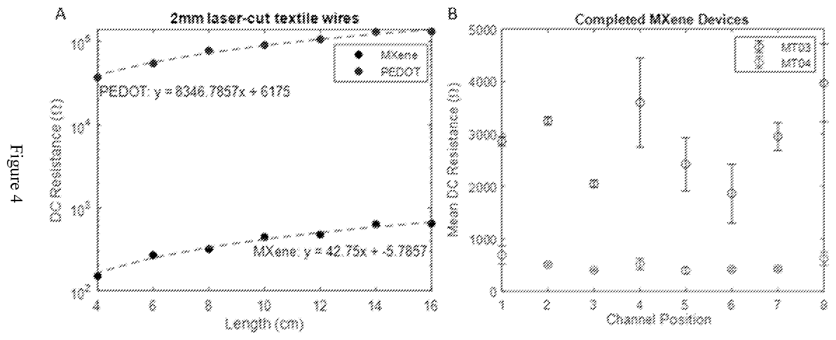

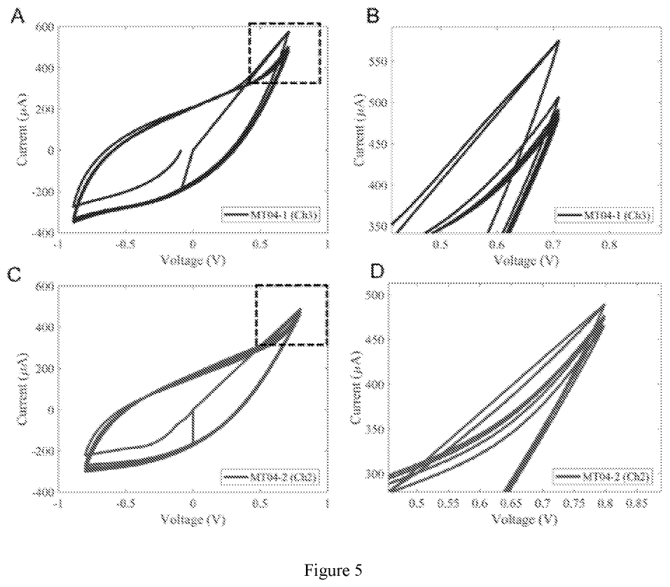

Cyclic voltammetry (CV) of MXene-textile electrodes suggests extremely high capacitance which is valuable for both neural stimulation and perhaps also energy storage (FIG. 5). Preliminary CV data suggest an electrode with high capacitance and reasonable anodic stability for a MXene electrode. Without being bound to any particular theory, it is possible that the higher surface area is leading to larger capacitive currents (i.e., lower capacitive impedance) and reduced Faradaic currents. A roughened matrix such as the absorbent materials can be a way to improve the electrochemical stability of MXene electrodes. Using CV, charge storage capacity (CSC) was calculated as the time integral of the cathodic current. An example of how cathodic and anodic regions of the CV plot are separated is shown in FIG. 6. MT04-1 exhibited cathodic CSC of 31.8 mC cm⁻² and MT04-2 exhibited a value of 28.2 mC cm⁻². These values are comparable to state-of-the-art stimulation materials, including iridium oxide (28.8 mC cm⁻²) and conductive polymers such as PEDOT (75.6 mC cm⁻²).

Electrochemical impedance spectroscopy (EIS) was performed on all 8 channels of both MXene-textile devices characterized in FIG. 5 and FIG. 6. The magnitude and phase are plotted in FIG. 7. An equivalent circuit model was fit to the data and is depicted in the inset of FIG. 7B. This is a modified version of the coated electrode model in which a film is covering a standard “Randles-like” electrode. In this model, accessing the electrode-electrolyte interface is only possible if there is a pore in the film thus leading to the pore resistance term (Rₚₒᵣₑ). The impedance variation between channels can (without being bound to any particular theory) be due to the different contact lengths of the MXene textile.

As a further comparison between MXene and PEDOT:PSS conductive inks, PEDOT-textile devices were created and EIS was performed in saline to compare electrode impedance between MXene-textile and PEDOT-textile devices. At the 1 kHz reference frequency, PEDOT-textile electrodes show an impedance of 5.27±1.53 kΩ, while MXene-textile electrodes show an impedance of 650.12±163.4 Ω. Thus, MXene-textile electrodes show a lower impedance compared to exemplary PEDOT-textile electrodes prepared according to the present disclosure.

### Acquisition of Surface EMG

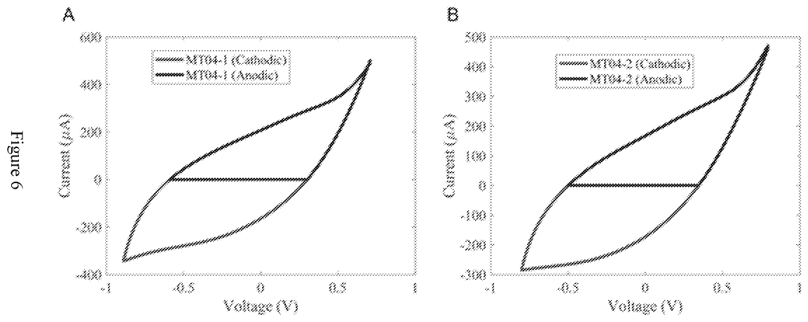

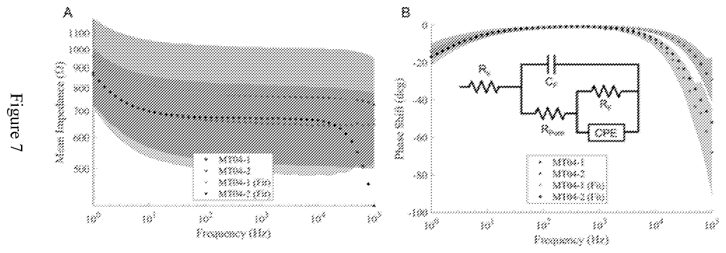

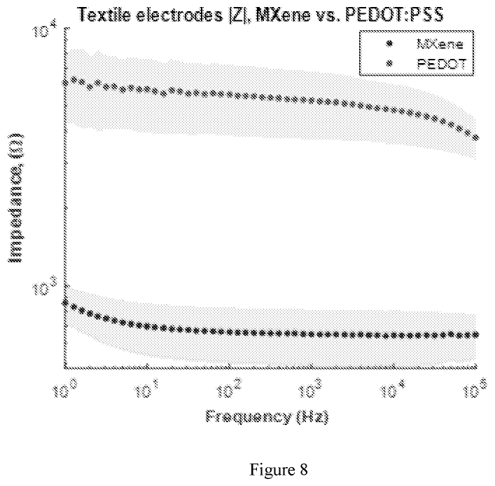

Surface EMG recordings were acquired using both of the MXene-textile devices described in this document: MT04-1 and MT04-2. The filtered (bandpass 80-350 Hz) recording traces, along with the force applied to the load cell, are shown in FIG. 8. Without being bound to any particular theory, the size of the EMG potential is seen to follow the force profile, and amplitude, of the load cell force trace.

### Protruding Electrodes

For a dry (gel-free) skin-based biosensor, intimate contact with the skin is essential for achieving low interface impedance and high-quality recording of biosignals. One simple modification to the above fabrication protocol was to form electrodes that protrude outward from the device. This was achieved by cutting 3 mm-diameter circles out of EYETEC cellulose eye spears using a biopsy punch, laying these over the electrode contact areas on the laser-patterned textile, and inking these at the same time that the laser-patterned textile is infused with the conductive ink. Upon wetting with the conductive ink, the cellulose sponge expanded upward, resulting in a conductive “pillar”. The device was then encapsulated in PDMS (or the polymer encapsulation of choice) and the pillars can be trimmed to the desired height.

FIG. 9A shows two images of an exemplary MXene-textile electrode array with the added cellulose sponge pillars after inking with MXene but prior to PDMS encapsulation. FIG. 9B shows several examples of electrode contacts in a finished device, after PDMS encapsulation and trimming of the conductive sponge pillars down to various heights.

These “pillar” electrodes represent a simple modification to the method described in this report, which broaden the applications achievable with these electrode arrays. These protruding pillar electrodes are useful for, e.g., obtaining electroencephalography (EEG) signals through hair, which can be a particular challenge for dry, gel-free electrodes. Arrays of pillar electrodes could be gently massaged/swirled into the head so as to get in between the hair and achieve good contact with the scalp.

### Conclusion

Provided here are, inter alia, methods for patterning absorbent materials with a laser cutter and then injecting conductive inks to create electrodes and interconnects via capillary action. These composite electrode arrays can be encapsulated in a variety of polymers.

As described, electrode contacts were exposed using a 3 mm diameter biopsy punch, though such exposure can be effected with other techniques. A variety of conductive-textile electrodes (using MXenes) were characterized electrochemically and exhibit excellent properties, including large charge storage capacity and low interfacial impedance. PEDOT-textile electrodes were also characterized. As an initial biosensing demonstration, MXene-textile devices were used to acquire surface EMG from a human subject in a ‘dry electrode’ paradigm in which no conductive gels were required. A simple modification to the fabrication method is also described, which enables the production of protruding ‘pillar’ electrodes which can be particularly useful for obtaining EEG recordings through the hair.

### MXene-Textile Dry EEG/Epidermal Electrodes

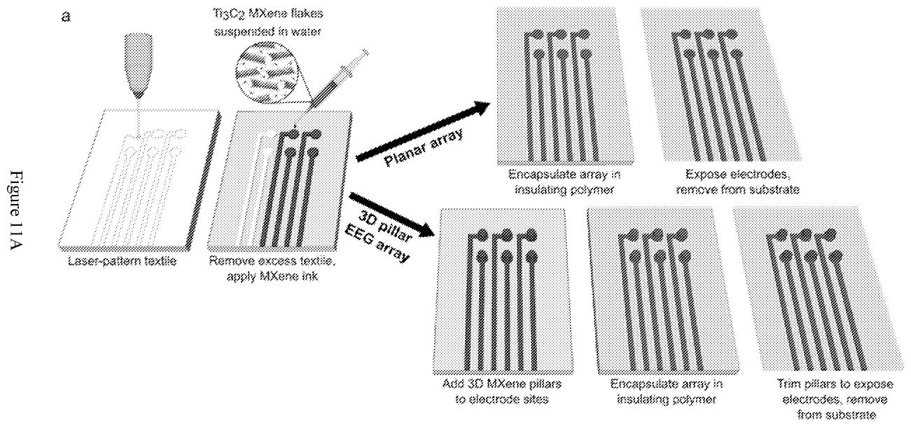

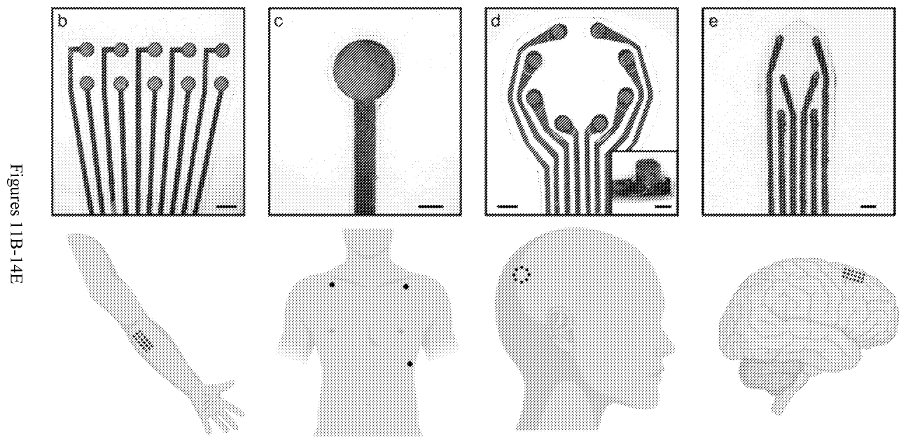

FIG. 14 provides an exemplary process for forming electrode arrays, as well as providing an image of a planar EMG sensing array fabricated using this method.

### EEG Recording on Human Subject Using Dry MXene-Textile Electrode Arrays

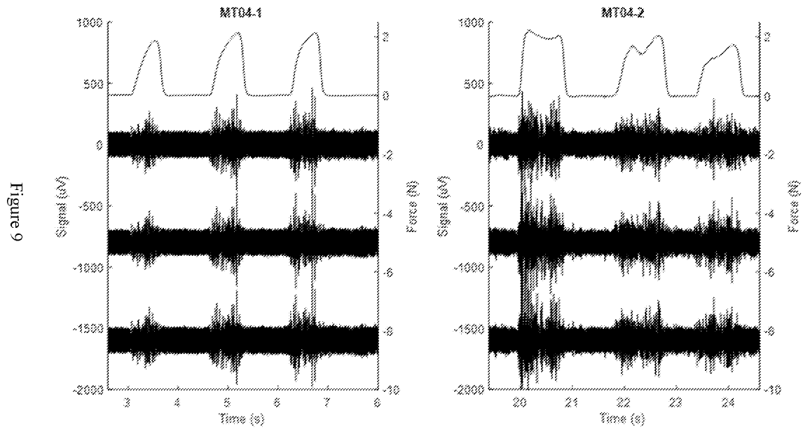

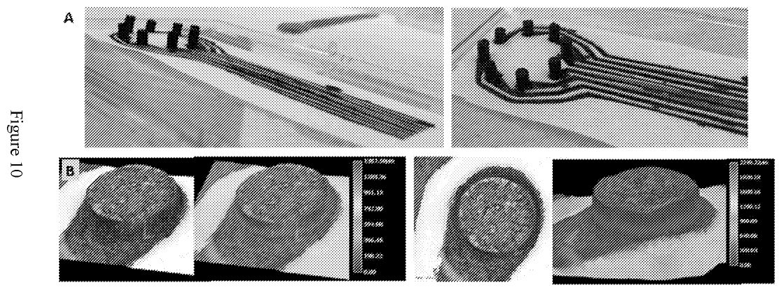

We have successfully demonstrated recording of high-fidelity EEG signals on 3 mm dry MXene-textile pillar electrode arrays on human subjects, comparing our data with simultaneously recorded EEG signal on a typical cm-scale gelled EEG cup electrode. Photos of the pillar electrode array and the recording setup on the human subject are shown in FIGS. 13a-13b.

To obtain quality EEG signals with minimal noise, an electrode-skin interface impedance of less than 10 kΩ at 1 kHz can be desirable. During a recording session with the MXene array and the gelled cup placed on the back of the head in a hairy region, the dry MXene-textile pillar electrodes showed 1 kHz impedance of 2.83±0.9 kΩ while the gelled cup showed a 1 kHz impedance of 1.21 kΩ (FIG. 2c). When these values are normalized by electrode geometric surface area (GSA), as is necessary to compare electrodes of different sizes, one finds that that the dry MXene-textile pillar electrodes actually exhibit significantly reduced impedance. EEG signals recorded in the occipital region of the human scalp while the subject was either resting with eyes open or eyes closed showed that the dry MXene-textile electrodes recorded EEG signals with comparable signal fidelity compared to the gelled cup electrode. (FIG. 13.) Clear increases in the power of the 10 Hz alpha rhythm across all electrodes during the eyes closed state was observed, as expected for the experimental paradigm. (FIG. 13.)

### MRI and CT Imaging Compatibility of MXene-Textile Electrode Arrays

Exemplary devices were (to show compatibility of MXene-textile electrode arrays with MRI and CT systems) imaged in agarose phantoms. For comparison to standard metallic electrodes, we imaged AdTec Platinum strip electrodes commonly implanted in epilepsy patients.

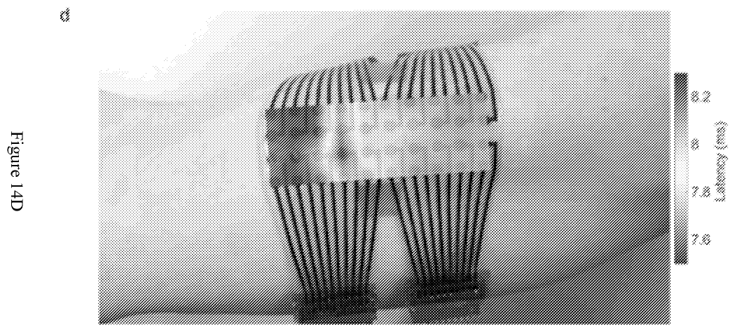

A schematic of the devices prepared for imaging is shown in FIGS. 18a-18c. In a 9.4T research-grade MRI machine, significant shadowing around the Pt electrodes, was observed. No shadowing, however, occurred around the MXene-textile electrodes (FIG. 18b). Similarly, in a high-resolution research CT scanner, significant X-ray scattering artifact around the Pt electrodes was observed, but no artifact visible from the MXene electrodes (FIG. 18c). In both imaging modalities, the conductive MXene-textile material is indistinguishable from the PDMS encapsulation material.

### Additional Disclosure

In this work, we demonstrate a new class of multichannel, high-density bioelectronic interfaces, which can be termed “MXtrodes” in some embodiments, which are capable of both high fidelity recording and effective stimulation of neural and neuromuscular circuits at multiple scales. The key value of the work presented here rests on a series of advances: first, we leverage the excellent processability of Ti₃C₂ MXene to develop a rapid, low-cost, and highly scalable method for fabricating multichannel electrode arrays of arbitrary size and geometry. Such a process is conducive to industrial manufacturing and paves the way for translating MXene bioelectronics into clinical and consumer markets. Second, we report the first comprehensive study of the electrochemical properties of Ti₃C₂ MXene relevant for recording and stimulation of bioelectronic circuits. We show that the electrochemical behavior of Ti₃C₂ MXene is comparable and in many cases superior to conventional bioelectronic materials, especially in the context of delivering charge for safe and effective stimulation. Third, we demonstrate the utility of MXtrodes for mapping and modulating excitable networks at scales ranging from humans to small animal models. In particular, we show gel-free multichannel arrays for large-scale human epidermal electronics with electrode-skin interface impedance and recording quality comparable to larger commercial single contact gelled Ag/AgCl electrodes. Furthermore, we demonstrate the ability of MXtrodes to finely map clinically relevant neural and neuromuscular activation patterns at high spatial and temporal resolution—which is elusive for conventional epidermal sensors—and to deliver effective stimulation. Fourth, we experimentally characterize the compatibility of Ti₃C₂ MXene with clinical imaging modalities and we demonstrate that MXtrodes minimally interact with magnetic fields and X-rays, resulting in artifact-free high-field MRI and computed tomography (CT) imaging. This finding opens new and exciting opportunities for future research and clinical paradigms combining high temporal resolution electrophysiology with advanced functional imaging. Propelled by the unique properties of Ti₃C₂ MXene and the high-throughput, scalable, and cost-effective fabrication process developed here, MXtrodes show great promise for numerous applications in healthcare, research, and wearable devices.

### Results

### Rapid, Low-Cost Manufacturing of MXtrodes

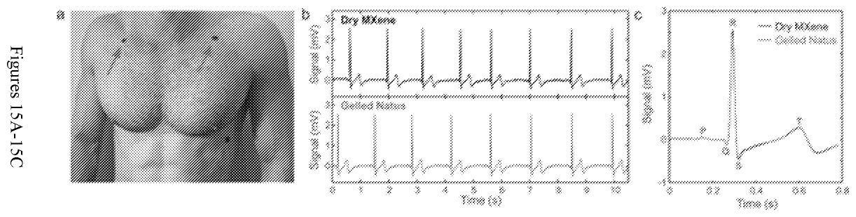

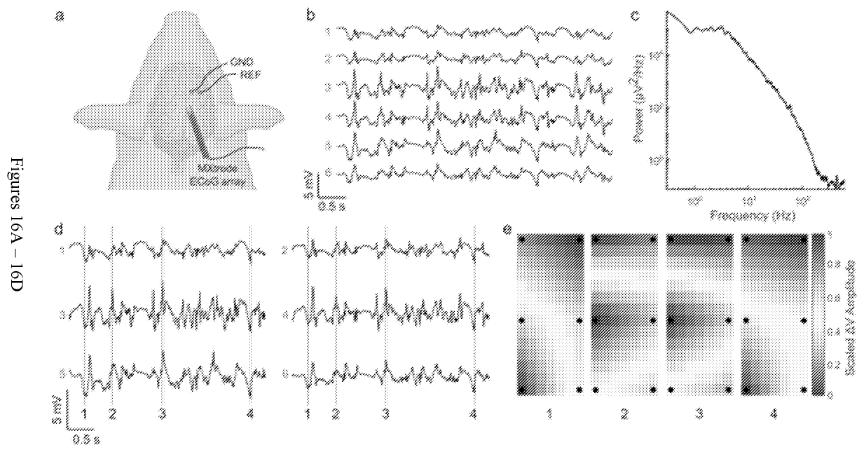

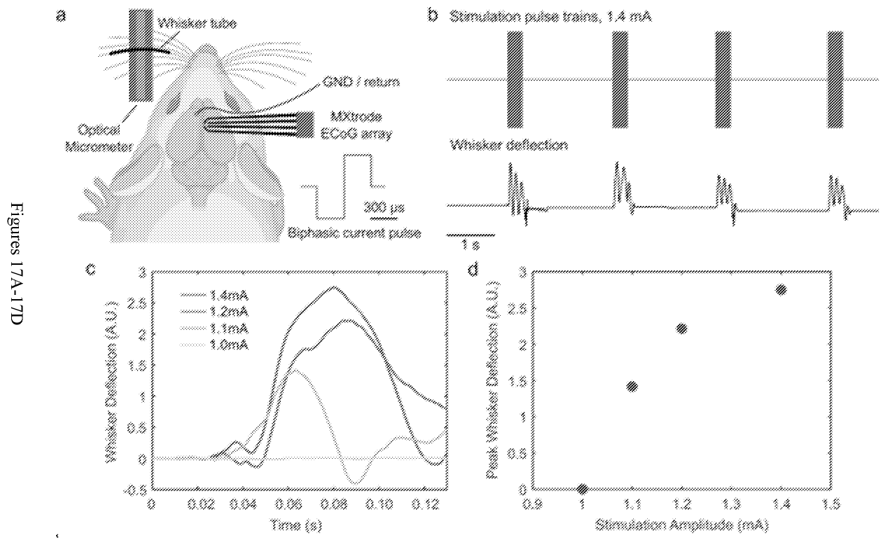

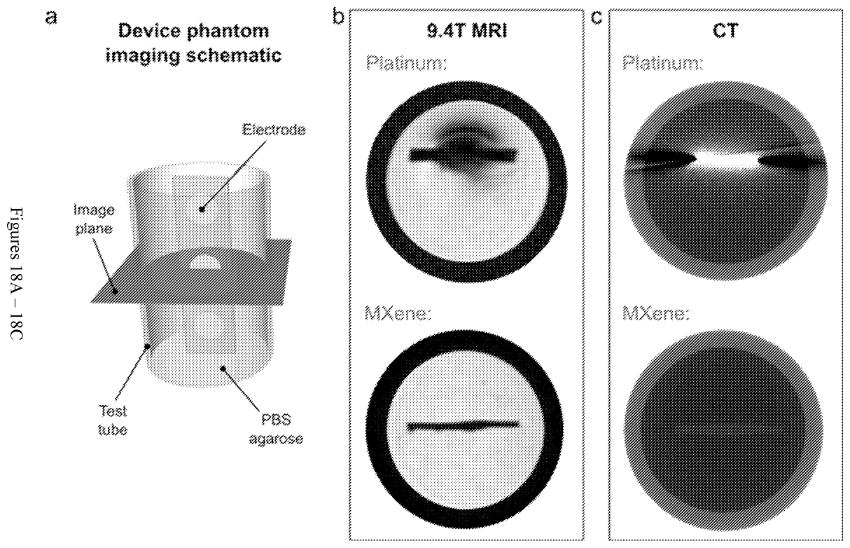

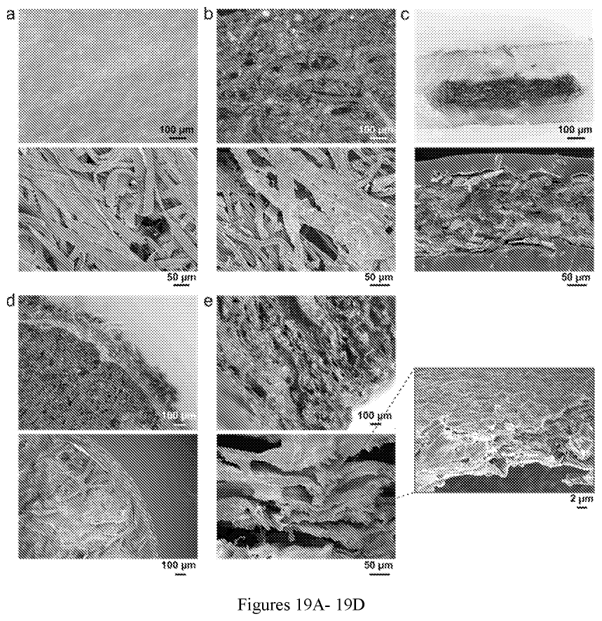

We developed a simple method for producing the MXtrode arrays, which involves laser-patterning a porous absorbent substrate, infusing it with a water-based Ti₃C₂ MXene ink, and then encapsulating the resulting conductive composite in flexible elastomeric films. For the various bioelectronic applications demonstrated in this work, we fabricated two different types of electrodes by slightly varying the same basic process: 1) flat or planar electrodes for epidermal sensing and for epicortical brain recording and stimulation, and 2) 3D “mini-pillar” electrodes for gel-free EEG recording. The versatility of our fabrication process allows addressing application-specific requirements and customizing the electrodes to the structures of interest: while for epidermal and cortical recording flat planar electrodes achieve adequate tissue coupling, gel-free EEG recording requires 3D components to overcome the hair barrier and make contact with the scalp. The fabrication process, with both variations, is depicted in FIG. 11a. Briefly, we used a CO₂ laser to pattern a nonwoven, hydroentangled cellulose-polyester blend substrate into the desired electrode array geometry. This served as a scaffold for the Ti₃C₂ MXene flakes, with the rapid laser patterning process allowing for rapid prototyping and customization of the array geometry. Next, we infused the cellulose-polyester substrate with Ti₃C₂ MXene ink, which was prepared by a minimally intensive layer delamination (MILD) method⁴⁵ to produce a water-based MXene ink with a concentration of 30 mg/mL. The ink quickly wicked into the absorbent substrate, coating all of the fibers. The ink-infused substrates were then dried in a vacuum oven for 1 hr at 70° C. and 60 mmHg to ensure that all water was removed. The resulting structure was a rough, macro-porous conductive composite, with MXene flakes coating each individual fiber in the textile matrix (FIGS. 19a, 19b). For the planar MXtrode arrays, the MXene conductive composite was then encapsulated in ˜1 mm-thick layers of polydimethylsiloxane (PDMS), with a thorough degassing step prior to curing allowing the PDMS to infiltrate into the conductive matrix (FIG. 19c). Electrode contacts were defined by manually cutting through the top encapsulation layer with a biopsy punch of the desired electrode diameter and peeling up the resulting PDMS disk to expose the conductive MXene composite beneath (FIG. 19d).

For the planar MXtrode arrays we used for cortical recording and stimulation, an additional 1 μm-thick layer of parylene-C was deposited prior to opening the electrode contacts to serve as an additional barrier to moisture. To fabricate 3D MXtrode arrays for gel-free EEG recording, “mini-pillars” of MXene-infused cellulose foam were deposited onto the electrode locations prior to PDMS encapsulation. Similar to the absorbent cellulose-polyester substrate, the cellulose foam readily absorbed the MXene ink such that it thoroughly coated all surfaces and formed a porous conductive composite after vacuum drying (FIG. 19e). It is worth noting that no adhesive was needed to affix the 3D pillars to the underlying laser-patterned substrate.

Inking the two structures simultaneously with MXene and vacuum drying while in contact allowed MXene to form a continuous conductive network fusing the laser-patterned substrate and the cellulose foam together. The final steps of the 3D MXtrode array fabrication involved PDMS encapsulation and manual trimming of the 3D pillars to expose the conductive MXene-cellulose foam composite. The versatility, simplicity, scalability, and low-cost of this process enabled parallel fabrication of MXtrodes in a variety of geometries for diverse bioelectronic applications, even within the same batch (see, e.g., FIGS. 11b-11e; FIG. 20).

### Electrical and Electrochemical Properties of MXtrodes

In the MXtrode arrays, the MXene-cellulose-polyester conductive composite forms the wires which carry the signal out to the recording amplifier. Thus, it is important that this composite is highly conductive to reduce ohmic losses, minimize noise, and acquire high-quality bioelectric signals. We measured the bulk conductivity of the MXene composites and determined it to be 3015.9 S/m (FIG. 21). To highlight the conductivity advantage of Ti₃C₂ MXene compared to other conductive inks—which could in principle be used with our fabrication process—we also fabricated conductive composites of PEDOT:PSS and reduced graphene oxide (rGO) inks with the same cellulose-polyester absorbent substrate. The bulk conductivity of these composites was 7.63 S/m and 0.005 S/m, respectively, significantly lower than MXene.

To evaluate the impedance and charge transfer properties of MXene electrodes and compare them with other common bioelectronic materials, we conducted electrochemical measurements on MXtrodes with diameters ranging from 500 μm to 3 mm, and compared to 2.3 mm-diameter clinical AdTech Pt electrodes. Specifically, we performed electrochemical impedance spectroscopy (EIS), cyclic voltammetry (CVs) and voltage transient experiments, which allowed us to measure the impedance magnitude, charge storage capacity (CSC), safe voltage window, and charge injection capacity (CIC) of each electrode, and to determine how these properties scale with electrode diameter. Data are shown in Table 1:

TABLE 1
Summary of electrochemical properties of planar MXtrodes of varying
diameters, compared to a 2.3 mm-diameter Pt electrode contact.

| | Electrode | | 1 kHz \|Z\| | Potential Limits | CSC_{C} | CIC_{C} |
|---|---|---|---|---|---|---|
| Material | diameter | | in PBS (Ω) | vs. Ag/AgCl (V) | (mC cm⁻²) | (mC cm⁻²) |
| MXene | 3 | mm | 205.56 ± 11.13 | −1.8-0.6 | 233.07 ± 19.64 | 0.551 ± 0.099 |
| MXene | 2 | mm | 369.20 ± 40.15 | −1.8-0.6 | 392.33 ± 9.80 | 0.690 ± 0.030 |
| MXene | 1 | mm | 430.2 ± 86.11 | −1.8-0.6 | 604.82 ± 61.39 | 1.072 ± 0.071 |
| MXene | 500 | μm | 729.18 ± 71.15 | −1.8-0.6 | 1024.13 ± 3.01 | 2.121 ± 0.481 |
| Pt | 2.3 | mm | 287.30 ± 7.84 | −0.6-0.8 | 4.34 ± 0.24 | 0.070 ± 0.003 |

Additional comparisons to other common electrode materials from literature data are tabulated in Table 2.

| | | 1 kHz \|Z\| | | CSC_{C} | CIC_{C} |
|---|---|---|---|---|---|
| | Geometric | in PBS (Ω) | Potential | (mC cm⁻²) | (mC cm⁻²) |
| | Surface | ↑ elec size = | Limits vs. | ↑ elec size = | ↑ elec size = |
| Material | Area (mm²) | ↓ \|Z\| | Ag/AgCl (V) | ↓ CSC | ↓ CIC |
| MXene | 7.069 | 205.56 ± 11.13 | −1.8-0.6 | 233.07 ± 19.64 | 0.551 ± 0.099 |
| MXene | 3.142 | 369.20 ± 40.15 | −1.8-0.6 | 392.33 ± 9.80 | 0.690 ± 0.030 |
| MXene | 0.7854 | 430.2 ± 86.11 | −1.8-0.6 | 604.82 ± 61.39 | 1.072 ± 0.071 |
| MXene | 0.1963 | 729.18 ± 71.15 | −1.8-0.6 | 1024.13 ± 3.01 | 2.121 ± 0.481 |
| Pt | 4.155 | 287.30 ± 7.84 | −0.6-0.8 | 4.34 ± 0.24 | 0.070 ± 0.003 |
| Pt | 3.142 | 1910 ^{a} | −0.6-0.8 | 3.6 ^{a} | 0.17 ^{a} |
| Au | 3.142 | 470 ^{a} | −0.9-0.6 | 0.3 ^{a} | 0.21 ^{a} |
| PEDOT:PSS on Pt | 3.142 | 1690 ^{a} | −0.9-0.6 | 2.3 ^{a} | 0.18 ^{a} |
| PEDOT:PSS on Au | 3.142 | 430 ^{a} | −0.9-0.6 | 1.0 ^{a} | 0.86 ^{a} |
| Pt | 0.1963 | 7790 ^{a} | −0.6-0.8 | 5.1 ^{a} | 0.50 ^{a} |
| Au | 0.1963 | 4430 ^{a} | −0.9-0.6 | 0.3 ^{a} | 0.21 ^{a} |
| PEDOT:PSS on Pt | 0.1963 | 2110 ^{a} | −0.9-0.6 | 3.7 ^{a} | 0.66 ^{a} |
| PEDOT:PSS on Au | 0.1963 | 990 ^{a} | −0.9-0.6 | 1.5 ^{a} | 1.35 ^{a} |
| laser-roughened Pt | 0.7854 | 760 ^{a} | −0.6-0.8 | 2.1 ± 0.1 | 0.026 ^{a} |
| PEDOT/CNT | 0.0314 | 2100 ^{a} | −0.6-0.7 | 70 | 2.5 ± 0.1 ^{a} |
| coating on Pt | | | | | |
| Porous graphene | 0.09 | 519 | −1.3-0.8 | 50 | 3.1 |
| (doped) | | | | | |
| CNT fiber | 0.00145 | 11.2 ± 7.6 × 10³ | −1.5-1.5 | 372 ± 56 ^{b} | 6.52 |
| TiN | 0.004 | 11.5 × 10³ ^{a} | −0.9-0.9 | 2.47 | 0.55 |
| IrOx | 0.004 | 7.1 × 10³ ^{a} | −0.6-0.8 | 11 | 4 |
| PtIr | 5.985 | 125 | −0.7-0.7 | 5.0 | — |

EIS revealed that the MXtrodes of all diameters tested showed significantly reduced impedance compared to the Pt electrodes at frequencies below 500 Hz, where most physiologic signals of interest lie (FIG. 12a). We attribute the exceptionally low impedance of the MXtrodes to the highly porous and rough morphology of the electrode surface, which gives it a high effective surface area.

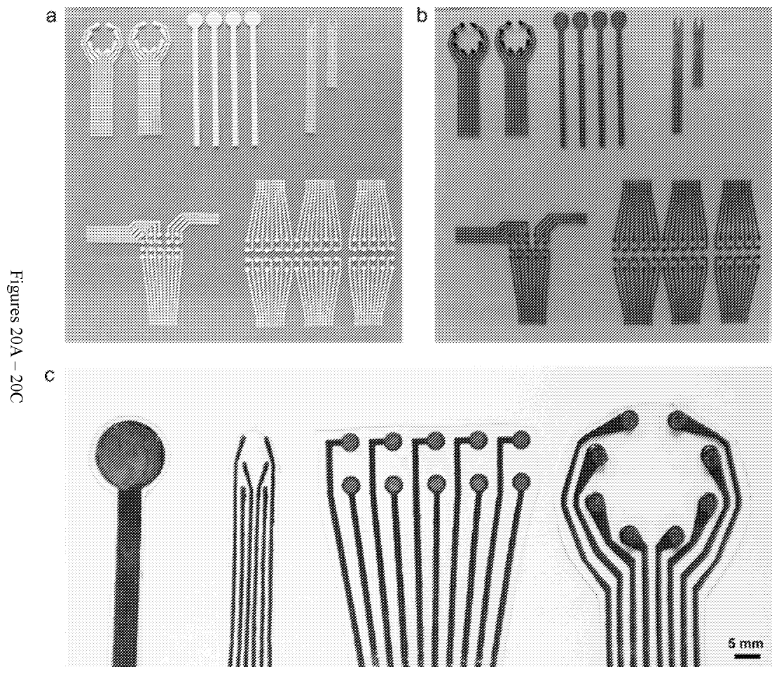

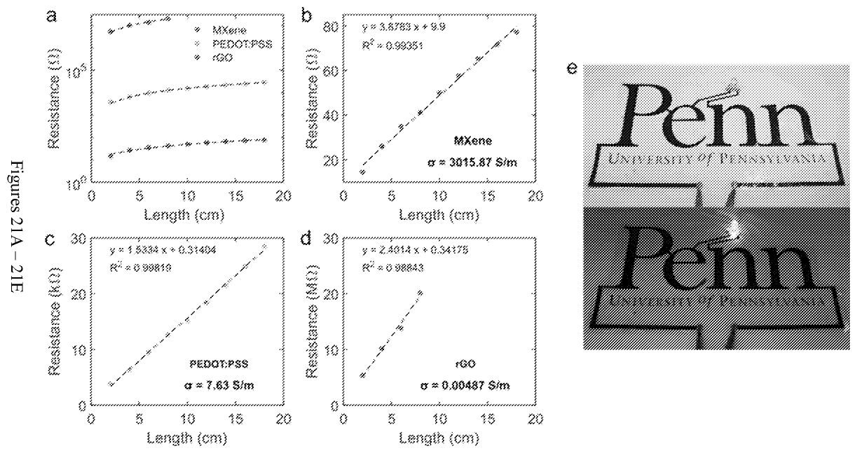

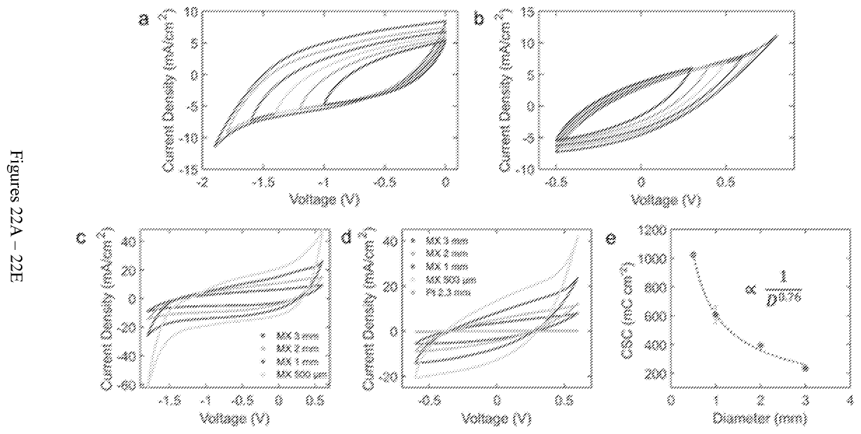

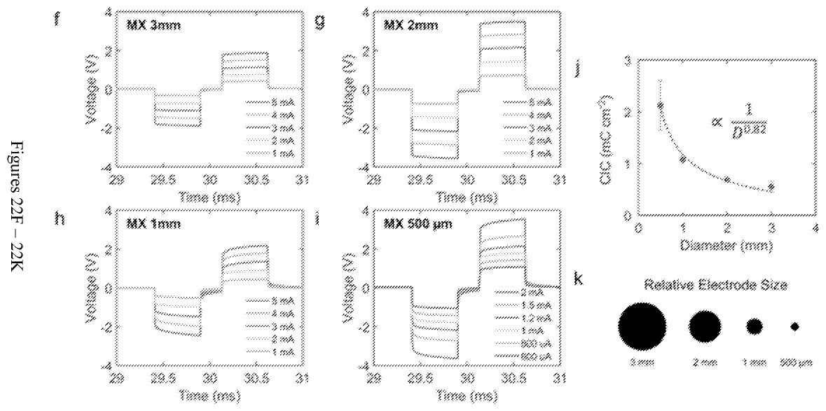

The safe voltage window for MXtrodes we determined from wide-scan CVs is −1.8-+0.6 V (FIGS. 22a-22b), showing that MXene is exceptionally stable in the cathodal region, with hydrolysis of water beginning at −1.9 V. This wide safe potential range is advantageous for therapeutic electrical stimulation applications, and while the safe anodal potential limit (+0.6 V) is slightly lower than that of Pt, PtIr, or IrOx, standard materials used for stimulation electrodes which all have an anodal limit of +0.8 V, stimulation pulse waveforms can be engineered to minimize voltage excursions in the anodal range while taking advantage of the large cathodal limit⁴⁶⁻⁴⁸. Analysis of cathodal CSC (CSC_{C}) for MXtrodes and Pt electrodes from CV at 50 mVs⁻¹ within their respective water windows reveals enhanced capacitive charging and charge delivery properties for MXtrodes with ˜100× larger CSC_{C} than same-sized Pt electrodes (Table 1, FIG. 22c). To enable a more direct comparison of CSC_{C} values between MXtrodes and Pt electrodes, we also ran CV scans in the intersection of the MXene and Pt voltage windows, −0.6-+0.6 V, as shown in FIG. 12b, FIG. 22d, and Table 3 below:

| | Electrode | | CSC_{C} (mC cm⁻²) | CSC_{C} (mC cm⁻²) | CSC_{C} (mC cm⁻²) |
|---|---|---|---|---|---|
| Material | diameter | | MXene window: −1.8-0.6 V | Intersection: −0.6-0.6 V | Pt window: −0.6-0.8 V |
| MXene | 3 | mm | 233.07 ± 19.64 | 80.87 ± 8.85 | N/A |
| MXene | 2 | mm | 392.33 ± 9.80 | 118.63 ± 10.10 | N/A |
| MXene | 1 | mm | 604.82 ± 61.39 | 188.46 ± 28.38 | N/A |
| MXene | 500 | μm | 1024.13 ± 3.01 | 288.66 ± 1.73 | N/A |
| Pt | 2.3 | mm | N/A | 3.9 ± 0.17 | 4.34 ± 0.24 |

Even in this more constrained voltage range, the MXtrodes show more than 20× enhanced CSC_{C} compared to Pt, which we attribute both to the exceptionally high intrinsic capacitance of Ti₃C₂MXene⁴⁹⁻⁵¹ and the high effective surface area of the MXtrode surface. The scaling dependency of CSC_{C} on electrode diameter for the MXtrodes is shown in FIG. 22e. The non-linear relationship between CSC_{C} and electrode diameter is expected, and reflects the known phenomenon of electrochemical charge exchange happening predominantly at the edge of the electrode^{48,52}.

Finally, we measured the voltage transients evolved on each electrode when used to deliver charge-balanced, cathodal-first biphasic current pulses ranging from 1 to 5 mA with duration of 500 μs in both phases (t_{c}, tₐ) and interpulse interval (tᵢₚ) of 250 μs (FIG. 12d and FIG. 22f-22i). For the 500 μm-diameter MXtrode, the current amplitude range was restricted to 600 μA-2 mA. The maximum cathodal excursion potential, E_{mc}, was taken 10 μs after the cathodal pulse end and CIC_{C} was defined as the injected charge at which E_{mc} would reach the water reduction potential. The resulting CIC_{C} values, shown in Table 1, reveal that the MXtrodes significantly outperform Pt electrodes, with MXtrodes showing ˜10× larger CIC_{C} than the Pt electrodes. This result has significant implications for stimulation applications, and suggests that MXtrodes can offer more efficient charge transfer than current state-of-the-art Pt electrodes, which could potentially prolong battery life for implantable simulation systems such as deep brain stimulation (DBS), vagal nerve stimulation (VNS), and cardiac pacemakers. The scaling dependency of CIC_{C} on electrode diameter for the MXtrodes is shown in FIG. 22j, again revealing the expected non-linear scaling dependency resulting from edge effects. A schematic depicting the relative sizes of the MXtrodes included in the analysis is shown in FIG. 22k.

While measurements in saline allow comparing the properties of MXtrodes to current standard electrode materials such as Pt, for epidermal sensing applications it is also essential to evaluate the impedance properties on human skin. Specifically, achieving low electrode-skin interface impedance is key for recording high-fidelity signals^{53,54} and becomes particularly challenging in gel-free structures like MXtrodes. Thus, we measured EIS for 3 mm-diameter MXtrodes, both in planar and 3D configuration, on clean human skin following standard preparation with an alcohol swab and light abrasion with 3M Trace Prep tape. The planar and 3D MXtrodes showed 1 kHz impedances of 6.62±2.87 kΩ and 4.92±2.64 kΩ, respectively, with the lower impedance of the 3D electrodes attributable to the improved contact from the protruding mini-pillars pressing into the skin (FIG. 12d). These impedance values, which can be normalized by their geometric surface area (GSA) of 0.071 cm² to 0.47±0.20 kΩ·cm² for planar and 0.35±0.12 kΩ·cm² for 3D MXtrodes, are among the best reported so far for dry, gel-free epidermal electrodes^{1,55,56} and ˜100× lower than those of commercially available gelled Ag/AgCl electrodes commonly used for electrodiagnostics and monitoring⁴².

### Epidermal Sensing of Bioelectronics Signals in Humans

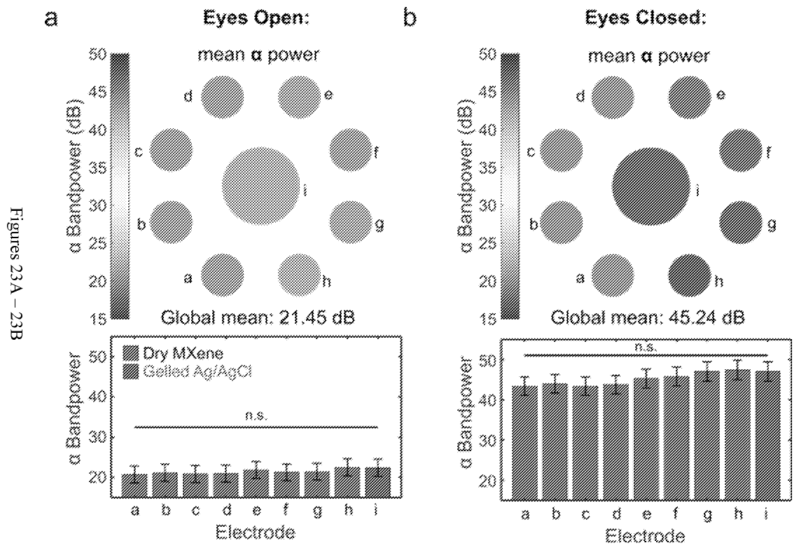

Motivated by the exceptionally low electrode-skin interface impedance of the dry MXtrodes, we investigated their use in a variety of epidermal sensing applications on human subjects, with custom geometries specifically designed for each application. First, we acquired scalp EEG on a healthy human subject using high resolution gel-free MXtrodes and standard gelled Ag/AgCl EEG electrodes for comparison. We designed an 8-channel MXtrode, with 3 mm-diameter 3D mini-pillar electrodes arranged in a ring around a central opening where a standard gelled Ag/AgCl EEG electrode could be placed for side-by-side comparison of simultaneously acquired EEG signals (FIG. 13a). In a first EEG experiment, we placed the MXtrode array over the parietal region near EEG site Pz and placed the gelled Ag/AgCl EEG electrode in the center (FIG. 13b), Ground and reference for all EEG recordings wereLelled electrodes placed at the center forehead and left mastoid, respectively. The subject had short hair (˜5 mm), and prior to placing electrodes the skin for the entire recording area was cleaned with an alcohol swab and lightly abraded with 3M Trace Prep tape. Notably, we found that the electrode-skin interface impedance at 1 kHz for the dry 3 mm-diameter MXtrodes was 2.83±0.91 kΩ, while the impedance of the 1 cm-diameter gelled Ag/AgCl electrode during the same experiment was 1.21 kΩ at 1 kHz (FIG. 13c). Given the critical role of the electrode-skin interface impedance in determining the quality of scalp EEG signals⁵⁷, most standard EEG electrodes require conductive gels at this interface, as well as a large contact area of at least ˜1 cm² to achieve suitably low impedance. Due to their enhanced material and surface area properties, mm-scale, gel-free MXtrodes can achieve strikingly low impedance, which enables high-resolution EEG recording. We recorded EEG in 2-min sessions, with the subject alternating between a resting state with the eyes closed and a resting state with the eyes open and fixating on a target. In both tasks, the EEG signal recorded on the dry MXtrodes was indistinguishable from the signal recorded on the gelled Ag/AgCl electrode (FIG. 13d). Additionally, a clear 10 Hz alpha rhythm emerged in the eyes closed state which was significantly larger in amplitude than in the eyes open condition (FIGS. 13e-13f). This alpha signature is one of the most reliable and widely studied behaviorally-linked EEG signatures in human subjects research⁵⁸, and arises from endogenous thalamic input to the visual cortex in the absence of visual input (i,e, when the eyes are closed)⁵⁹. When we calculated the alpha bandpower in is windows with 0.5 s overlap across the length of the recording session for each electrode, we observed no significant difference between the mean alpha power recorded on the gelled Ag/AgCl electrode and any individual dry MXtrode, confirming that the signals were comparable between the electrode types (FIG. 23). Interestingly, when these alpha bandpower values are observed sequentially, distinct spatiotemporal patterns of alpha activation emerge across the sampled scalp area, highlighting the potential of high-density EEG mapping enabled by mm-scale, gel-free MXtrodes.

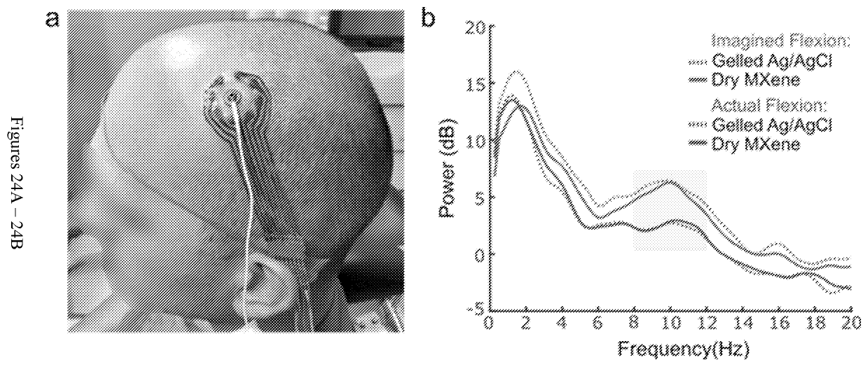

For a second EEG task, the electrodes were removed and replaced at site C3 near the hand motor area. The precise location of the hand motor area was determined using single pulses of transcranial magnetic stimulation (TMS) to evoke finger movements, and the electrodes were centered over this location (FIG. 24a). Skin was prepped as previously described. The subject performed 2-min periods of imagined and actual hand flexion while the EEG was recorded simultaneously with the co-located MXtrodes and gelled Ag/AgCl electrode. During this motor task, we also found that the EEG signal was indistinguishable between the two electrode types. Furthermore, we observed a suppression of the 8-12 Hz motor mu rhythm during the actual hand flexion relative to the imagined hand flexion (FIG. 24b). This signature mu suppression during actual vs. imagined hand movement is an important EEG feature which has been successfully used as a control signal for EEG-based brain computer interfaces (BCIs)^{60,61}. The results of these EEG experiments confirm that gel-free, mm-scale MXtrodes can record EEG signal at least as well as standard gelled Ag/AgCl EEG electrodes, while also improving spatiotemporal resolution for high-density EEG applications.

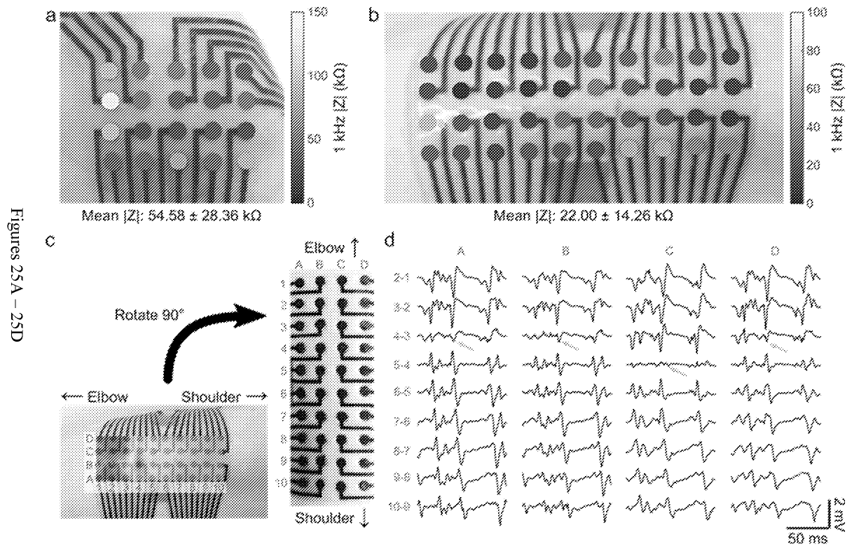

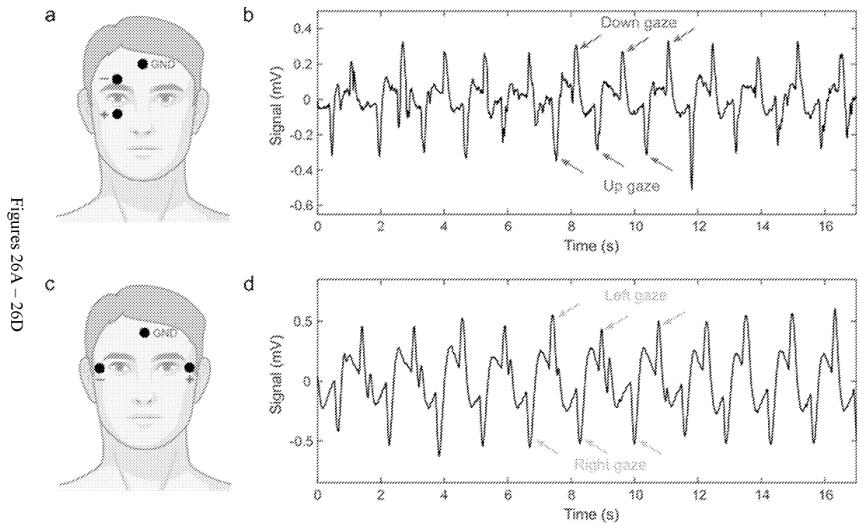

Next, we evaluated MXtrodes for high-density surface EMG (HDsEMG) recording, muscle activation mapping and precise localization of the of innervation zones (IZs). HDsEMG is attracting growing interest for a number of applications in neuromuscular diagnostics and rehabilitation, including control of multifunctional prostheses¹⁸, studies of muscle activation and coordination⁶², peripheral nerve/muscle fiber conduction velocity measurements⁶³, and for accurate localization of neuromuscular junctions (NMJs) to target chemodenervation therapies for muscle spasticity^{64,65}. HDsEMG recordings require flexible, large-area, and high-density electrode arrays capable of covering the wide range of muscle sizes. To demonstrate the versatility of the MXtrode fabrication process, we created custom HDsEMG arrays and used them to map muscle activation and localize IZs in two muscle groups of different sizes (FIG. 14a-14d). First, we used a 20-ch planar MXtrode array placed over the abductor pollicis brevis (APB) at the base of the thumb, following a standard skin prepping protocol. In this experiment, the 3 mm-diameter dry MXtrodes had an average electrode-skin interface impedance of 54.6±28.4 kΩ at 1 kHz (FIG. 25a). We then stimulated the median nerve with a handheld bipolar stimulator probe to evoke APB contractions and recorded the EMG on the MXtrode array. We calculated the mean evoked muscle response across stimulation trials (FIG. 14a) and constructed a latency map of the peak of the evoked response. The location with the shortest latency indicates the location of the IZ, which can be seen overlaid on the subject's hand in FIG. 14b.

Following the APB mapping experiment, we mapped the activation of the larger biceps brachii with a 40-ch planar MXtrode array. Here, the 3 mm-diameter dry MXtrodes had an average electrode-skin interface impedance of 22.0±14.3 kΩ at 1 kHz (FIG. 25b). Unlike the smaller APB muscle, which has a small and spatially confined IZ corresponding to a single NMJ, the larger biceps brachii has distributed NMJs in IZ regions that run perpendicular to the muscle and are typically located near the center⁶⁶. To confirm the validity of our approach, we used two different methods to localize these IZs in the biceps: first we stimulated the supraclavicular nerve and constructed a latency map of the peak evoked response, similar to the methods described for the APB (FIGS. 14c-14d). This produced a clear mapping of the IZ location as the region with the shortest latency, running across the short head of the biceps brachii. Second, we recorded motor unit action potentials (MUAPs) as the subject performed isometric contractions of the biceps (FIGS. 25c-25d). In these recordings, bipolar subtraction of the raw EMG signal along the length of the biceps muscle revealed MUAPs that propagated outward in both directions from the IZ, with signal inversion and a clear latency as the MUAP traveled away from the IZ (FIG. 25d). Localization of the biceps IZ obtained from both methods were in excellent agreement. These results demonstrate that dry, high-resolution MXtrode arrays are capable of mapping muscle activation with high accuracy in order to precisely localize IZs in both small and large muscles.

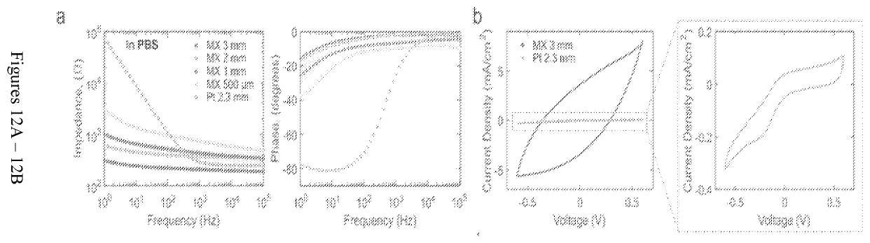

To demonstrate the applicability of MXtrodes to electrocardiogram (ECG), we acquired ECG recordings on a healthy human subject with 1.3 cm-diameter MXtrodes in a simplified 3-electrode montage as shown in FIG. 15a. For validation and signal comparison, we recorded sequentially from the MXtrodes and from 1 cm-diameter pre-gelled commercial Ag/AgCl electrodes, placed in the same locations. The dry MXtrodes and pre-gelled electrodes had average 1 kHz skin-electrode impedances of 1.29 kΩ and 1.38 kΩ, respectively. On both types of electrodes, characteristic ECG features were clearly visible, with the P wave followed by the QRS complex and the T wave (FIG. 15b-15c). Mean R peak amplitude, however, was slightly higher on the MXtrodes compared to the commercial electrodes: 2.55±0.06 mV vs. 2.47±0.30 mV. Thus, we confirm that dry MXtrodes can record the ECG with comparable signal quality compared to standard gelled electrodes.

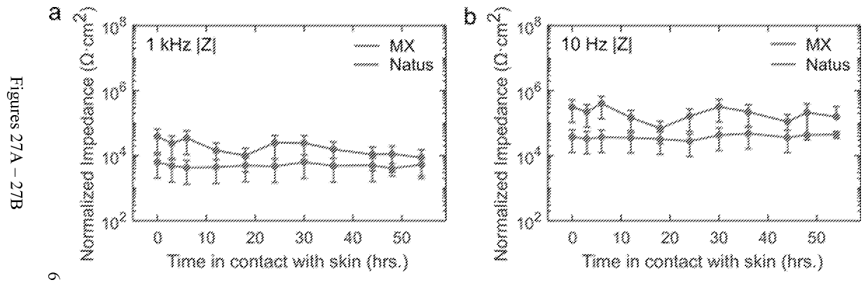

As a final demonstration of MXtrodes for human epidermal sensing, we acquired electrooculography (EOG), which has applications in ophthalmological diagnosis, for human-machine interfaces, and for monitoring attention and fatigue^{67,68}. The EOG signal arises from the standing dipole potential between the positively charged cornea and the negatively charged retina, which enables tracking eye movements as this dipole is rotated. With the same 1.3 cm-diameter dry MXtrode geometry used in ECGs, we recorded the EOG in two configurations to track up-down and left-right eye movements (FIG. 26). By placing MXtrodes above and below the eye, recorded voltage fluctuations could be decoded to track the up and down movements of the eye (FIG. 26a-26b). Similarly, placing MXtrodes on both sides of the eyes enables decoding the left-right movements of the eyes (FIG. 26c-26d).

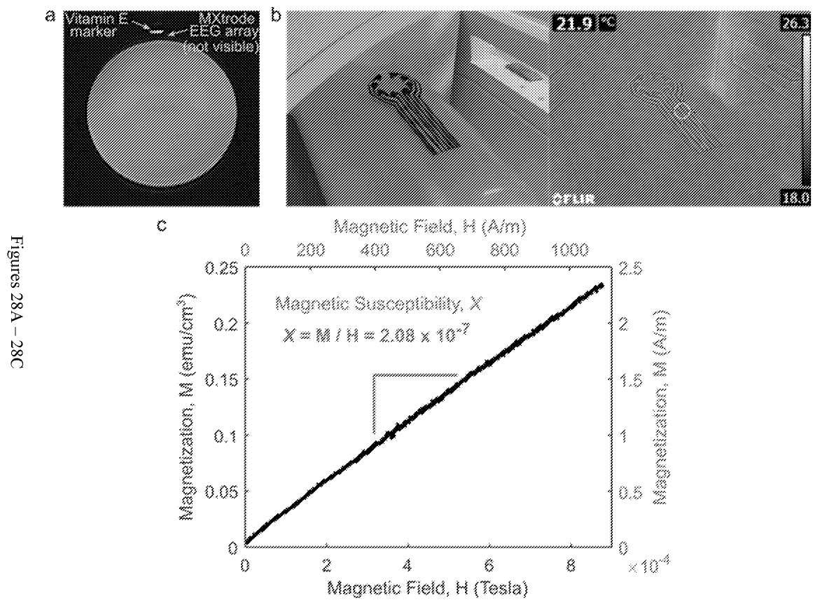

While we have demonstrated the MXtrodes for a range of acute epidermal sensing applications here, gel-free electrodes are also ideal for chronic monitoring and integration in wearable sensors for long-term use. For such applications, it is critical that the electrodes exhibit stable impedance behavior over time on the skin. Thus, we monitored the electrode-skin interface impedance for arrays of 3 mm-diameter planar MXtrodes as well as 1 cm-diameter pre-gelled commercial electrodes on healthy human subjects for up to 54 hrs. We observed a slight decrease in impedance magnitude for both the MXtrode arrays and the pre-gelled electrodes over the full 54 hrs, which we attribute to sweat uptake. At 1 kHz, the impedance of the MXtrodes went from 552.1±377.08 kΩ initially to 123.51±89.41 kΩ at 54 hrs. Similarly, at 1 kHz the impedance of the pre-gelled electrodes went from 8.07±5.50 kΩ initially to 6.70±4.17 kΩ at 54 hrs. We show the area-normalized impedance values over time at both 1 kHz and 10 Hz in FIG. 27.

### Neural Recording and Stimulation with MXtrodes

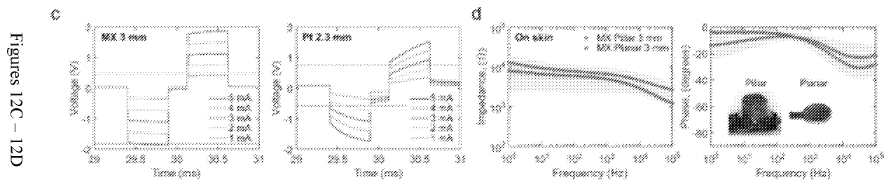

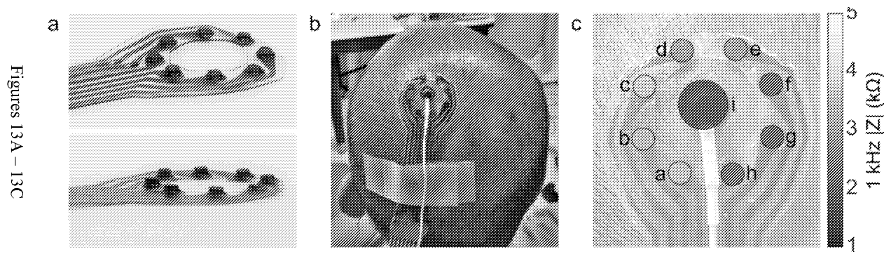

Beyond epidermal sensing modalities, the favorable electrochemical interface of the MXtrodes also support their use for implantable sensing and stimulation applications. One such application is intraoperative electrocorticography (ECoG) recording, a common mapping technique used in resective brain surgery for epilepsy or tumors. We acquired ECoG recordings in an anesthetized swine, a relevant model system in neuroscience given its gyrencephalic structure and neuro-anatomical similarity to the human brain. In this experiment we inserted a 6-ch array of 500 μm-diameter planar MXtrodes through an 8-mm craniotomy/durotomy and placed MXtrodes in direct contact with the cortical surface (FIG. 16a). The array configuration consisted of 3 rows of electrode pairs with 5 mm inter-row spacing and 4.5 mm pitch so that the rows of electrodes spanned several cortical gyri. A few seconds of representative raw ECoG signal are shown in FIG. 16b. The signals were large in amplitude with negligible 60 Hz noise interference, as evidenced by the power spectra (FIG. 16c). Furthermore, maps of interpolated voltage across the MXtrode array revealed stereotyped spatial patterns emerging during the “up” and “down” states in the ECoG signal (FIGS. 16d-16e), highlighting the advantages and opportunities offered by high-density cortical brain mapping with MXtrodes.

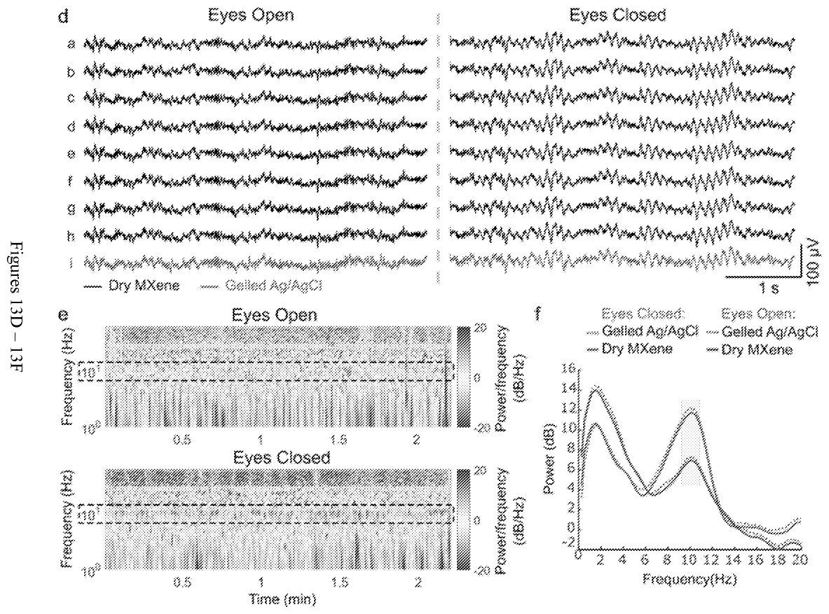

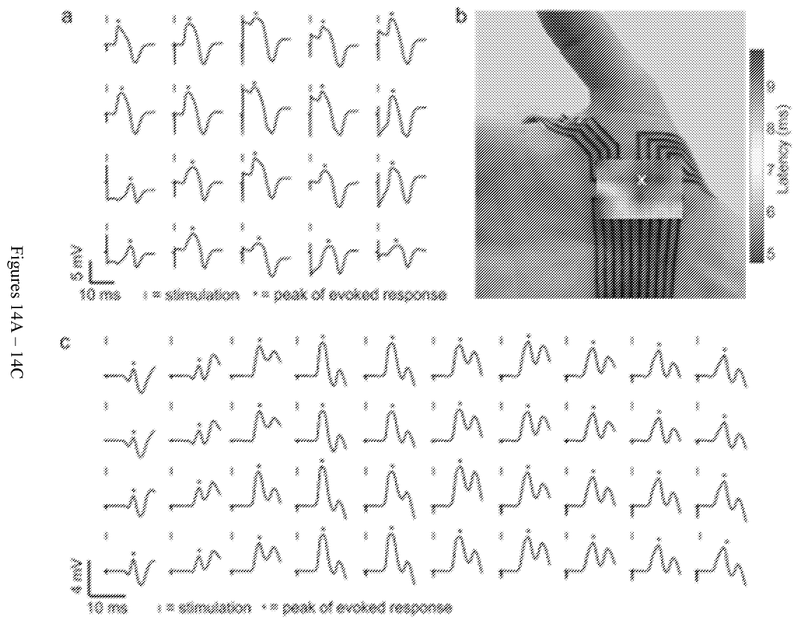

In addition to ECoG recording, direct stimulation of the cortical surface is used clinically for intraoperative cortical mapping⁶⁹ and neuromodulation therapies⁷⁰, as well as for closed-loop BCIs⁷¹. Given that the MXtrodes showed superior CSC and CIC compared to Pt, a material commonly used in stimulating electrodes, we sought to demonstrate the effectiveness of MXtrodes for electrical stimulation by evoking motor responses via intraoperative stimulation in rat brain. Specifically, we placed a 500 μm-diameter planar MXtrode epidurally onto the sensorimotor cortex of an anesthetized rat. Contralateral to the MXtrode, an optical micrometer was positioned to track whisker displacement, with one whisker placed into a plastic tube to maximize detection sensitivity (FIG. 17a). Trains of charge-balanced, cathodal-first stimulation pulses with amplitudes ranging from 1.0 to 1.5 mA were delivered through the MXtrode. We observed stimulation-evoked whisker movements for amplitudes greater than 1.0 mA, with the whisker deflection amplitude scaling with the stimulation intensity (FIGS. 17b-17d). Whisker movements registered as oscillatory deflections on the micrometer, with the first deflection peak always being the largest in amplitude. To compare whisker deflection across stimulation amplitudes, we computed the mean amplitude of the first whisker deflection peak for each stimulation trial for a given stimulation amplitude. This experiment confirms that MXtrodes are capable of delivering electrical stimulation to effectively modulate neural activity.

### Compatibility of MXtrodes with Clinical Imaging

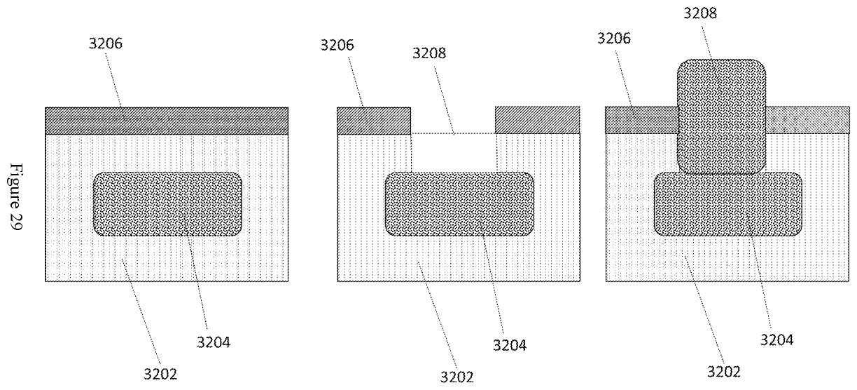

With the widespread adoption of bioelectronic technologies, the compatibility of these devices with clinical imaging has become increasingly important. MRI and CT are the two most common imaging techniques used in the diagnosis of injury and disease as well as in image-guided interventions. Many of the conductive materials traditionally used in bioelectronic devices are incompatible with the challenging MRI environment, and can produce heating or exert forces on the tissues. Even devices considered MR-safe often produce imaging artifacts that shadow the surrounding anatomical structures, caused by a mismatch in magnetic susceptibility between the device and the surrounding tissue⁷². These challenges are compounded at high field strengths, which are seeing increasing use for high resolution imaging and novel functional and metabolic imaging techniques^{73,74}. While the magnetic susceptibility of Ti₃C₂ MXene had not been previously reported, we hypothesized from the weak dia- and paramagnetic properties of C and Ti respectively that MXene can have a low magnetic susceptibility and thus prove compatible with the MR environment. To verify our hypotheses, we performed MRI scans of MXtrodes and measured the magnetic properties of Ti₃C₂ MXene at body temperature. First, we imaged cross-sections of 3 mm-diameter planar MXtrodes and 2.3 mm-diameter commercial Pt electrodes embedded in conductive agarose phantoms in a 9.4T high-field MRI system (FIG. 18a-18b). In the MR images, we found significant shadowing around the Pt electrodes, while no artifact was visible around the MXtrode (FIG. 18b). In fact, the MXene composite forming the conductive electrode was almost completely indistinguishable from the surrounding PDMS encapsulation. To further explore the MRI compatibility of the MXtrodes, we next imaged an array of 3 mm-diameter 3D mini-pillar MXtrodes, the same array used for EEG recording in this work, in a 3T clinical MRI scanner. The MXtrodes were placed atop an MRI phantom and imaged using a battery of scan sequences. Regardless of the scan sequence, the MXtrodes showed no artifact and were nearly invisible in the MRI images (FIG. 28a). Thermal IR images of the MXtrode array immediately following a 10 min scan sequence also showed no evidence of heating of the electrode array (FIG. 28b). Finally, we measured the magnetic susceptibility of Ti₃C₂ MXene at 310 K, and found it to be 2.08×10⁻⁷ (FIG. 28c), indicating that Ti₃C₂ MXene is weakly paramagnetic with magnetic susceptibility very close to that of human tissues (−11.0 to −7.0×10⁻⁶). This innate property of Ti₃C₂ MXene, which has not been reported previously, leads directly to the excellent compatibility of the MXtrodes with MRI imaging. For comparison, the magnetic susceptibility of Pt is 2.79×10⁻⁴, several orders of magnitude larger than the susceptibility of human tissues⁷². Regarding CT compatibility, the x-ray attenuation characteristics from the high density of many conductive materials pose challenges for CT imaging with bioelectronic devices⁷⁵. Ti₃C₂ MXene has a density of 3.7 g/cm³, which is ˜5 times lower than Pt, thus we hypothesized that MXene could minimize attenuation and scattering artifacts in CT⁷⁶. To test our hypothesis, we imaged the same 3 mm-diameter planar MXtrodes and 2.3 mm-diameter commercial Pt electrodes embedded in conductive agarose phantoms that we imaged in the 9.4 T MRI system. We observed significant x-ray scattering artifacts around the Pt electrode, but not around the MXtrode (FIG. 18c).

### Discussion

The simple fabrication method reported here offers a scalable and low-cost means of producing large-area, multichannel bioelectronic interfaces which can record and modulate the activity of excitable tissues across multiple scales. The method is conducive to large-scale manufacturing, a key aspect for translation beyond the lab and into clinical and consumer markets, and it also enables rapid customization of MXtrode array geometries for different bioelectronic applications and even for achieving patient- or subject-specific fit where desired. The exceptional properties of Ti₃C₂ MXene endow these electrodes with impedance and charge delivery properties which meet or exceed current state-of-the-art electrode materials in both implantable and epidermal use cases. In epidermal sensing applications, the low electrode-skin interface impedance of the gel-free MXtrodes opens up exciting new possibilities for high-resolution EMG and EEG, while eliminating the challenges associated with wet conductive gels. With HDsEMG, MXtrode arrays can allow accurate localization of NMJs, which can eliminate the need for the painful and invasive needle EMG procedures commonly used today to target chemodenervation therapies for spasticity. Such HDsEMG arrays can also prove useful for advanced prosthetic limb control, where EMG recordings from the residual limb are a useful control signal^{77,78}. In EEG applications, a dry electrode system enabled by MXtrodes can offer a route toward minimizing skin breakdown and alleviating many of the key logistical challenges associated with current gelled EEG systems, such as the time required to apply each electrode and impedance fluctuations over time as gels dry out. These advantages, coupled with the possibility for obtaining high density EEG recording with mm-scaled MXtrodes, also make this technology attractive for neuroscience research and for non-invasive BCI systems. The compatibility of MXtrodes with both CT and MRI imaging modalities is also a highly desirable attribute, allowing subjects to receive imaging procedures without necessitating removal of electrodes and without device-induced artifacts in the resulting images. This imaging compatibility further opens exciting possibilities for multimodal studies, such as those combining simultaneous EEG with fMRI or GluCEST imaging^{74,79}. For implantable electrode applications, the electrochemical properties of the MXtrodes, particularly those relating to charge delivery, are quite striking. The high CSC and CIC compared to Pt suggests that MXtrodes can offer an alternative material for stimulation electrodes and can enhance the efficiency of charge transfer to prolong battery life of implantable stimulation systems. MXene-based bioelectronic interfaces show exciting potential to enable the next generation of wearable and implantable devices to improve healthcare diagnostics and monitoring, and to enable new capabilities in wearable devices and multimodal imaging and electrophysiology studies. While challenges remain, such as improving the oxidation-resistance of Ti₃C₂ MXene to enable long-term applications, significant progress has already been made⁸⁰⁻⁸² which opens the door to future studies using MXene-based bioelectronics.

### Methods

### Synthesis of Ti₃C₂ MXene

Ti₃C₂ MXene was produced using the MILD synthesis method⁴⁵ to create an ink of 30 mg/mL Ti₃C₂ MXene in DI, which was placed in a vial and sealed in Argon. The average size of the Ti₃C₂ MXene flakes was 4 μm in lateral dimension.

### Fabrication of MXtrode Devices

Devices were fabricated by first laser-patterning absorbent nonwoven textile substrates comprised of hydroentangled 55% cellulose/45% polyester blend (Texwipe TechniCloth) using a CO₂ laser (Universal Laser Systems PLS 4.75) such that electrode array patterns were easily separable from the surrounding textile but could still be lifted and handled as one sheet. These were transferred to a thin and slightly tacky bottom layer of 1:10 PDMS (Sylgard 184) on a flat acrylic sheet, and the excess textile surrounding the array patterns was then peeled up.

The textile patterns were then inked with 30 mg/mL Ti₃C₂ MXene in DI, allowed to air dry for 15 mins, then placed in a vacuum oven (Across International) at 70° C. and 700 mmHg for 1 hr to remove all remaining water. For devices incorporating 3D pillar electrodes, 3 mm circles were cut from absorbent cellulose sponges (EyeTec Cellulose Eye Spears) using a 3 mm biopsy punch and these circular sponges were inked with MXene and placed onto the electrode locations of the MXene-textile constructs at the same time that the laser-patterned textile was inked.

The subsequent drying steps were identical. No adhesive was necessary at this interface, as the dried MXene and the subsequent PDMS encapsulation was sufficient to hold the 3D pillars firmly in place. For multichannel arrays, connectors (FCI/Amphenol FFC&FPC clincher connectors) were then attached by screen printing silver conductive epoxy (CircuitWorks CW2400) onto the ends of the dried MXene constructs, inserting these into the connectors, and clinching shut. The devices were baked at 70° C. for 30 min to cure the silver epoxy. Next, 1:10 PDMS was layered over the devices to form the top insulation layer, thoroughly degassed at 700 mmHg for 15 mins, which forced PDMS to infiltrate into the MXene composite matrix, and cured at 70° C. for 1 hour. Finally, devices were cut out with a razor blade, and peeled up from the acrylic substrate. For planar electrodes, electrode contacts were exposed by cutting circular holes though the top PDMS layer using a biopsy punch (diameters ranging from 3 mm-500 μm) and carefully peeling up the disk of PDMS to expose the MXene composite electrode below. For 3D pillar electrodes, electrode contacts were exposed by trimming the tops of the 3D pillars with a flat razor blade, exposing the MXene-sponge composite electrode. Slight variations on this method were utilized for MXtrode arrays designed for different applications: for EMG arrays, a thin layer of silicone medical adhesive spray (Hollister Adapt 7730) was applied to the skin-facing side of the array prior to opening the electrode contacts to enhance skin adhesion; for single-channel ECG and EOG MXtrodes, EcoFlex (Smooth-on Ecoflex 00-30) in a 1:2 ratio (part A:part B) was used as the encapsulation rather than PDMS to offer enhanced skin adhesion and comfort; for the ECoG electrodes, arrays were fabricated in PDMS as described above, but were additionally coated in a 3 μm-thick layer of Parylene-C prior to opening electrode contacts to enhance the moisture barrier properties of the encapsulation.

### Imaging of MXtrode Devices

Optical images of MXtrodes and their constituent parts were taken with a Keyence VHX6000. Scanning electron microscopy images were captured using a Zeiss Supra 50VP Scanning electron microscope with an accelerating voltage of 5 kV.

### DC conductivity of Ink-Infused Composites

DC conductivity measurements were made on laser-cut test structures 20 cm long×3 mm wide×285 μm thick comprising 55% cellulose/45% polyester blend (Texwipe TechniCloth) infused with either: (1) 30 mg/mL Ti₃C₂ MXene in DI, (2) 1.1% high conductivity grade PEDOT:PSS in H₂O (Sigma Aldrich), or (3) 18 mg/mL highly concentrated single-layer graphene oxide (Graphene Supermarket) which was subsequently reduced using a vitamin-C reduction method⁸³. Measurements were taken with a handheld multimeter with flat alligator clip terminations, where the negative lead was fixed at the end of the construct and the positive lead was moved in 2 cm increments for each measurement.

### Electrochemical Characterization of MXtrodes

For skin EIS measurements, the skin of the inner forearm was prepared with an alcohol swab followed by light abrasion (3M TracePrep) before placing 3 mm planar and pillar MXtrodes and measuring EIS from 1-10⁵ Hz with 10 mVₚₚ driving voltage using a Gamry Reference 600 potentiostat. Reference was placed on the inner wrist and ground was placed on the elbow (Natus disposable disk electrodes). Electrochemical measurements in saline, including EIS, CV, and current pulsing, were performed for planar MXtrodes with diameters of 3 mm, 2 mm, 1 mm, and 500 μm, and Pt electrodes with a diameter of 2.3 mm (Adtec epilepsy subdural grid TG48G-SP10X-000) in 10 mM PBS (Quality Biological) using a Gamry Reference 600 potentiostat. EIS was measured from 1-10⁵ Hz with 10 mVₚₚ driving voltage. Cyclic voltammetry was performed at a sweep rate of 50 mVs⁻¹. Safe voltage limits for MXtrodes were determined by incrementally increasing the negative limit of the CV scan until water reduction was observed (beginning at −1.9 V), then the positive limit of the CV scan until a linear, resistive behavior was observed (beginning at +0.7 V) beyond which the MXtrode showed current loss with subsequent scans. CSC, was determined from the CV scans by taking the time integral of the cathodal current.

Current pulsing was performed using chronopotentiometry with biphasic, charge-balanced current pulses with t_{c}=tₐ=500 μs and tᵢₚ=250 μs for currents ranging from 600 μA to 5 mA for N=3 electrodes of each size. For CIC_{c} calculations, E_{mc} was determined as the instantaneous voltage 10 μs after the end of the cathodal current pulse. E_{mc} values were plotted as a function of injected current amplitude, and the linear relation was determined to estimate the current limit at which the electrode would reach its cathodal limit (−1.8 V for MXtrodes, −0.6 V for Pt). Any series of measurements in which current amplitude vs. E_{mc} was not linear with R²<0.95 were excluded. CIC, was defined as: CIC=Iₗᵢₘ×t_{c}/GSA where Iₗᵢₘ is the cathodal current limit, t_{c} is the cathodal pulse width, and GSA is the electrode geometric surface area. CSC and CIC scaling relations were determined by fitting power functions to the data, similar to work by M. Ganji et al⁸⁴.

### EEG Experiments

EEG experiments were conducted under a protocol approved by the Institutional Review Board of Drexel University (Protocol #1904007140). The healthy human subject was seated in a comfortable chair with a head rest. Prior to placing electrodes, the subject's scalp was prepared with an alcohol swab and light abrasion (3M TracePrep), though the presence of hair may have limited the efficacy of the skin abrasion. Recordings were made using an 8-electrode MXtrode device with dry 3 mm-diameter 3D pillar electrodes and 1 standard gelled Ag/AgCl EEG cup electrode (Technomed Disposable EEG cup) placed in the center of the MXtrode array. Recordings were obtained using a NeuroNexus SmartBox amplifier system with a sampling rate of 20 kHz. Standard gelled Ag/AgCl EEG cup electrodes were used for reference (placed on left mastoid) and ground (placed on forehead center). In the first set of recordings, electrodes were positioned on the parietal region, centered over P1. Pre-wrap (Mueller) was used to hold the electrodes in place, and a small hole was made in this wrapping to allow application of conductive gel (SuperVisc, EASYCAP GmbH) beneath the standard EEG cup electrode. 6, 2-minute-long recordings were obtained, alternating between eyes open and eyes closed states. For the eyes open state, the subject was asked to fixate on a cross on a computer monitor. In the second set of recordings, the hand motor region was localized using single TMS pulses and the electrodes were positioned centered over this location, near site C3. 6, 2-minute-long recordings were obtained, cycling through a resting state, imagined hand flexion, and actual hand flexion. Signals were notch filtered at 60 Hz and bandpass filtered from 0.1-100 Hz.

### EMG Experiments

EMG, ECG, and EOG human epidermal recordings were conducted under an experimental protocol approved by the Institutional Review Board of the University of Pennsylvania (Protocol #831802). For all EMG experiments, skin preparation prior to placing MXtrode arrays included an alcohol swab followed by light abrasion (3M TracePrep), and signals were recorded at a sampling rate of 20 kHz on an Intan RHS2000 Stimulation/Recording Controller (Intan Technologies).

For recordings from abductor pollicis brevis (APB), a 5×4 grid of 3 mm planar MXtrodes with center-to-center spacing of 7.5 mm horizontal, 6.5 mm vertical, was placed over the APB at the base of the thumb. Reference was placed over the bony interphalangeal thumb joint and ground was placed on the back of the hand (Natus disposable adhesive electrodes). The median nerve was stimulated using a VikingQuest handheld bipolar stimulator (Nicolet), starting at 10 mA and gradually increasing until clear activation of APB was observed in the form of a thumb twitch (amplitude 38.8 mA for the subject shown).

For localization of the NMJ of the APB, evoked responses recorded on each electrode in the array (no signal filtering was used) were averaged across all stim trials (N=10), the peak of the average evoked response was determined, and a map of the latency of this peak from the onset of the stimulation was created. The location of the NMJ was approximated as the location with the shortest latency in the peak evoked response. For recordings of the biceps, a 10×4 grid of 3 mm planar MXtrodes with center-to-center spacing of 8.5 mm horizontal, 8.5 mm vertical, was placed over the center of the biceps muscle. Reference was placed distal to the array just above the inner elbow and ground was placed proximal to the array on the deltoid (Natus disposable adhesive electrodes). The supraclavicular nerve was stimulated using the same VikingQuest handheld bipolar stimulator (Nicolet), starting at 30 mA and gradually increasing until clear activation of biceps was observed (amplitude 49.0 mA for the subject shown). As for the APB recordings, evoked responses recorded on each electrode were averaged across all stim trials (N=11), the peak of the average evoked response was determined, and a map of the latency of this peak from the onset of the stimulation was created. The IZ was determined as the area with the shortest latency in the peak evoked response.

To further validate the IZ localization in the biceps, additional EMG recordings were made with the subject performing periods of isometric voluntary contractions. Signals were subtracted in bipolar configuration down the length of the MXtrode array, and MUAPs were identified in the raw EMG signal. The IZ was determined as the region where the MUAP appears earliest and where the signal polarity inverts.

### ECG Recording

For ECG recording experiments, skin preparation prior to placing electrodes included an alcohol swab followed by light abrasion (3M TracePrep), and signals were recorded at a 20 kHz sampling rate on an Intan RHS2000 Stimulation/Recording Controller (Intan Technologies). Recordings were made in a 3-electrode configuration with reference placed just below the subject's right clavicle, ground placed just below the subject's left clavicle, and the working electrode placed on the lower left ribs. For comparison, recordings were made sequentially using either all 2 cm gelled Natus electrodes (Natus disposable adhesive electrodes) or all 1.3 cm dry MXtrodes, with electrodes placed in the same locations.

### ECoG Recording

Pigs were pair housed when possible, and were always in a shared room with other pigs in a research facility certified by the Association for Assessment and Accreditation of Laboratory Animal Care International (AAALAC facility). All experiments were conducted according to the ethical guidelines set by the Institutional Animal Care and Use Committee of the University of Pennsylvania and adhered to the guidelines set forth in the NIH Public Health Service Policy on Humane Care and Use of Laboratory Animals (2015).

Prior to the procedures, animals were fasted for 16 hours with water remaining ad libitum. After induction with 20 mg/kg of ketamine (Hospira, 0409-2051-05) and 0.5 mg/kg of midazolam (Hospira, 0409-2596-05), anesthesia was provided with using 2-2.5% isoflurane (Piramal, 66794-013-25) via a snout mask and glycopyrrolate was given subcutaneously to curb secretions (0.01 mg/kg; West-Ward Pharmaceutical Corp., 0143-9682-25). The animals were intubated with a size 6.0 mm endotracheal tube and anesthesia was maintained with 2-2.5% isoflurane per 2 liters O₂. Animals were then moved to an operating room, where they were transferred onto a ventilator. The ventilator provided the same rate of isoflurane and O₂ for anesthesia maintenance at a breath count of 20-25 breaths per minute. Heart rate, respiratory rate, arterial oxygen saturation, end tidal CO₂, blood pressure and rectal temperature were continuously monitored, while pain response to pinch was periodically assessed. All of these measures were used to maintain an adequate level of anesthesia. A forced air warming system was used to maintain normothermia.

Prior to electrode insertion, the pig was placed in a stereotaxic frame described previously (Ulyanova et al., 2018), and the surgical site was draped and prepared. After the skull was exposed, an 11 mm craniectomy was performed recording site, 7 mm lateral to midline and 4.5 mm posterior to bregma in order to expose the pig frontoparietal cortex. The dura was resected to expose the cortical surface allowing for subdural recording with the MXtrode array. Recordings with the MXtrode grid were obtained using an HS-36 amplifier, and collected continuously at 32 kHz using a Neuralynx Digital Lynx SX recording system. Raw data was collected and stored using Neuralynx's Cheetah recording software.

### Neural Stimulation

Stimulation pulses were delivered through each MXtrode to identify which one was optimally placed over the motor cortical region for eliciting whisker movements. Stimulation pulses were biphasic with t_{c}=tₐ=300 μs and zero interpulse interval, with currents ranging from 1.0 to 1.4 mA. Whisking movements were detected using an optical micrometer, with a tube placed over one whisker to amplify the signal.

### Evaluation of MRI and CT Compatibility

A strip of six 3 mm-diameter MXtrodes were prepared with PDMS encapsulation to match the geometry of the comparison Pt clinical ECoG electrode strips (Adtec epilepsy subdural grid TG48G-SP10X-000). Both types of electrode arrays were placed in 0.6% agarose (IBI Scientific) prepared with 10 mM PBS (Quality Biological) in a 15 mm inner-diameter glass test tube, with degassing to remove air bubbles. A 9.4 T Horizontal bore MRI scanner (Bruker, Erlangen) and 35 mm diameter volume coil (m2m Imaging, USA) were used to acquire T1-weighted gradient echo MR images of cross-sections of both electrode types. The acquisition parameters for T1-W MRI were: TE/TR=7/150 ms, FOV=30×30 mm, Matrix size=256×256, Averages=4, Flip angle 30°, slice thickness=0.7 mm. For CT imaging, a μC.T50 specimen scanner (Scanco Medical, Bruttisellen, Switzerland) was used to scan the electrodes at 70 kV, 115 μA, and 10 μm isotropic resolution. 2D images in the axial plane of each electrode type were acquired for comparison.

### Magnetic Susceptibility Measurements

Magnetic property was measured with Quantum Design Evercool 2 physical property measurement system. Free-standing film of Ti₃C₂Tₓ with a mass of 4.820 mg was packed in a plastic sample container. The sample was heated to 310 K and was allowed to reach thermal equilibrium for about 10 min. Magnetization was recorded with respect to the applied magnetic field up to 9 Tesla. The measured data was subtracted from that of the plastic sample holder and normalized by sample mass.

### Exemplary Embodiments

The following Embodiments are illustrative only and do not limit the scope of the present disclosure or of the claims appended hereto.

Embodiment 1. A component, comprising: (a) one or more sensors, a sensor comprising: (i) a permeable substrate material having an upper surface, the permeable substrate optionally being non-conductive; and (ii) an electrically conductive material, the electrically conductive material disposed within and/or on the permeable substrate material so as render the permeable substrate material conductive; and (b) an insulating material, the insulating material having an upper surface and a thickness, and the insulating material defining at least one aperture extending through the thickness of the insulating material, the at least one aperture being in register with a sensing location on the upper surface of the permeable substrate material of a sensor.

A permeable material can be an absorbent material, e.g., a textile. The conductive material can be affixed (e.g., bound, linked, attracted) to the permeable material, e.g., via electrostatic, ionic, or covalent bonds or other interactions. For example, the conductive material and the permeable material can both by hydrophilic.

Embodiment 2. The component of Embodiment 1, wherein the electrically conductive material comprises a MXene material, graphene, graphene oxide, graphite, carbon black, carbon nanotubes, nanoparticles (e.g., metallic nanoparticles), a conductive polymer, or any combination thereof. Metal from an electroplating solution can also be reduced to solid metal form and used as an electrically conductive material. It should be understood that the foregoing are examples only and do not limit the electrically conductive material that can be used.

As but one example, a strip of a fibrous and/or porous material can be infused with a conductive material (e.g., MXene flakes or graphene) to form a conductive trace. The infused strip (conductive trace) can be encased in an insulating material, e.g., PDMS, polyethylene, or other such material. A user can the form an aperture (which can be circular, but can also be polygonal or elongate in shape) in the insulating material so as to expose a sensing region of the conductive trace. The sensing region can be below the top surface of the insulating material.

As described elsewhere herein, the sensing region can be in electronic communication with a monitor or other device configured to collect a signal from the sensing portion. Also as described elsewhere herein, a conductive extension (e.g., a conductive pillar or other structure) can be placed into electrical communication with (or even contacted to) the sensing region through the aperture. As shown in the appended figures, e.g., FIG. 11A, the exposed region of the sensing region can be disposed at an end of the conductive trace, and the conductive extension can extend essentially perpendicular to the conductive trace. The conductive extension can physically contact the sensing region, but this is not a requirement, as an additional material or materials—such as a conductive sealant or adhesive—can be used to place the conductive extension into electronic communication with the sensing region.

The extension can have a surface that is flush or even with the uppermost surface of the insulating material (or materials) in which the sensing region is disposed. The extension can also have a surface that is above the uppermost surface of the insulating material (or materials) in which the sensing region is disposed.

A variety of example embodiments are shown in FIGS. 11A-11E. As shown, a device can include a plurality of sensor regions, i.e., exposed regions of conductive material. A device can be configured such that a first line drawn in a plane connects two or more sensor regions that line along that first line, and a second line in a plane connects two or more sensor regions that lie along that second line; such a configuration is shown in FIG. 11B. Alternatively, a device can be configured such three or more sensors line along a circular line drawn in a plane. As shown, a device can be configured such that the device comprises a proximal end and a distal end, with different sensing regions lying at different distances as measured from the distal end.

Embodiment 3. The component of any one of Embodiments 1-2, wherein at least two of the one or more sensors do not physically contact one another. As an example, two of the sensors can comprise parallel strips of permeable material, which strips do not contact one another. As described elsewhere herein, individual sensors can be individually addressable and in electronic isolation from one another.

Embodiment 4. The component of any one of Embodiments 1-3, wherein at least one sensor comprises a curved portion.

Embodiment 5. The component of any one of Embodiments 1-4, wherein at least some of the sensing locations of the one or more sensors define a periodic array that lies in a plane.

Embodiment 6. The component of any one of Embodiments 1-4, wherein the sensing locations of the one or more sensors define a circle that lies in a plane.

Embodiment 7. The component of any one of Embodiments 1-6, further comprising a conductive extension contacting and extending from the sensing location of a sensor through the aperture of the insulating material so as to extend beyond the upper surface of the insulating material.

Embodiment 8. The component of Embodiment 7, wherein the conductive extension comprises the electrically conductive material. In some embodiments, however, the conductive extension can comprise an electrically conductive material that differs from the electrically conductive material of the sensor from which the conductive extension extends.

A variety of materials can be used as conductive extensions, e.g., conductive rubbers, hydrogels, plated metals, casted inks, conductive fabrics, and the like. For example, carbon black or platinum impregnated rubbers can be used. MXene-infused cellulose structures are also suitable for use as conductive extensions.

Embodiment 9. The component of any one of Embodiments 1-8, wherein at least two of the one or more sensors are individually electronically addressable.

Embodiment 10. The component of any one of Embodiments 1-9, wherein a sensor is characterized as having a variable cross-sectional dimension. A sensor can be configured for deployment on a subject's limb, on the subject's scalp, on an organ, on the brain, or on another part of the subject's anatomy.

Embodiment 11. The component of any one of Embodiments 1-10, wherein the insulating material comprises a polymer. Example, non-limiting polymers include, e.g., silicones (e.g., PDMS), EcoFlexTM, polyurethane, polyimides, epoxy resins (e.g., flexible such resins), PEEK, polystyrene, elastomers, polyimide, and the like.

Embodiment 12. The component of any one of Embodiments 1-11, wherein the permeable substrate material comprises a woven textile. As mentioned elsewhere herein, absorbent substrates are considered suitable.

Embodiment 13. The component of any one of Embodiments 1-11, wherein the permeable substrate material comprises a non-woven textile.

Embodiment 14. The component of any one of Embodiments 1-11, wherein the permeable substrate material comprises a porous material.

Embodiment 15. The component of any one of Embodiments 1-14, further comprising a sealant conformally disposed on the insulating material. Parylene-C is one exemplary such sealant; other example sealants include (without limitation), spray-coated silicones, elastomers, epoxies, paraffins and the like.

Embodiment 16. The component of any one of Embodiments 1-15, further comprising an electrocatalytic element in electronic communication with the sensing location.

Example electrocatalytic elements include (without limitation), a conductive ink with dissolved metal nanoparticles (e.g., Au or Pt), carbon nanomaterials (e.g., nanotubes or graphene flakes), and electroplated electrocatalyst films. Without being bound to any particular theory, these elements can serve two purposes for electrochemical sensing: (1) enabling detection of inner sphere redox species, such as dopamine, which require on-surface adsorption; (2) reducing the overpotential required to oxidize or reduce a chemical species of interest.

Embodiment 17. The component of any one of Embodiments 1-16, further comprising a biosensing element in electronic communication with the sensing location. As but one example, one can use an enzyme (e.g., glutamate oxidase) that oxidizes glutamate and generates an electroactive substance (e.g., hydrogen peroxide); the electroactive substance is then oxidized or reduced to produce a measurable current. A biosensing element can be, e.g., an aptamer or antibody used in impedance-based sensing modalities. For example, antigen binding events increase or decrease the electrochemical impedance. An example of linking biomolecules to cellulose is described in J. Mater. Chem. B, 2013,1, 3277-3286, the entirety of which article is incorporated herein by reference.

Embodiment 18. The component of any one of Embodiments 1-17, wherein the component is configured for implantation into a subject.

Embodiment 19. A method, comprising: collecting a signal with a component according to any one of Embodiments 1-18. Such methods can include, without limitation, the use of such components in EEG, ECG, EMG, ECoG, and/or neural stimulation monitoring. The methods can include, without limitation, placing a sensor of a component in electronic communication with the skin, scalp, brain, or muscle of a subject. A signal can be collected while the component is at least partially within an MRI device or a CT device.

Such a signal can be, e.g., an electrical signal. The signal can be related to a voluntary action of a subject (e.g., movement of a limb, blinking, reading, watching a video). The signal can also be related to an involuntary action of the subject (e.g., breathing, involuntary eye movement, involuntary muscle contraction).

Embodiment 20. A method, comprising: fabricating a component according to any one of Embodiments 1-18.

Embodiment 21. A device, the device comprising: one or more components according to any one of Embodiments 1-18.

Embodiment 22. The device of Embodiment 21, wherein the device is characterized as being an electromyography (EMG) device, an electroencephalogram (EEG) device, an electrocardiogram (EKG) device, an electrocorticogram (ECoG) device, a skin conductivity device, a body area network device, a strain sensor, a pressure sensor, a temperature sensor, a skin conductivity sensor, an electrostimulation device, an implantable sensing or stimulation device, a chemical sensor, or any combination thereof

Embodiment 23. A method, comprising: infusing a fluid that comprises a carrier and a conductive material into a permeable substrate portion and then removing at least some of the carrier, the infusing and removing being carried out under such conditions that the conductive material renders the permeable substrate conductive; disposing an electrically insulating material over the permeable substrate, the electrically insulating material having an upper surface and defining a thickness; optionally disposing a sealant over the electrically insulating material; forming an opening through the thickness of the electrically insulating material (and through the sealant, if present), the opening being in register with a sensing location on the permeable substrate.

Embodiment 24. The method of Embodiment 23, further comprising patterning the permeable substrate portion from a larger portion of the permeable substrate.

Embodiment 25. The method of Embodiment 24, wherein the patterning comprises laser cutting, mechanical cutting, mechanical etching, chemical etching, or any combination thereof

Embodiment 26. The method of any one of Embodiments 23-25, wherein the conductive material is characterized as being hydrophilic.

Embodiment 27. The method of any one of Embodiments 23-26, wherein the fluid is aqueous.

Embodiment 28. The method of any one of Embodiments 23-26, wherein the fluid is non-aqueous.

Embodiment 29. The method of any one of Embodiments 23-26, wherein the fluid is organic.

Embodiment 30. The method of any one of Embodiments 23-29, wherein the electrically insulating material comprises a polymer, the polymer optionally being elastomeric.

Embodiment 31. The method of any one of Embodiments 23-30s, wherein the electrically insulating material comprises PDMS.

Embodiment 32. The method of any one of Embodiments 23-31, wherein the sealant comprises Parylene-C.

Embodiment 33. The method of any one of Embodiments 23-32, wherein the conductive material comprises, e.g., MXene material, graphene, graphene oxide, graphite, carbon black, a metal, a conductive polymer, or any combination thereof. Other such suitable materials are described elsewhere herein.

Embodiment 34. The method of any one of Embodiments 23-33, wherein the permeable substrate portion comprises cellulose, polyester, or any combination thereof

Embodiment 35. The method of any one of Embodiments 23-34, further comprising disposing a conductive extension that contacts and extends from the sensing location through the aperture of the insulating material so as to extend beyond the upper surface of the insulating material.

Embodiment 36. The method of any one of Embodiments 23-35, wherein the method is performed in a continuous manner.

Embodiment 37. The method of any one of Embodiments 23-36, wherein the method is performed in a batch manner.

Embodiment 38. A component, comprising: (a) one or more sensors, a sensor comprising: a conductive permeable substrate material having an upper surface; and (b) an insulating material, the insulating material having an upper surface and a thickness, and the insulating material defining at least one aperture extending through the thickness of the insulating material, the at least one aperture being in register with a sensing location on the upper surface of the permeable substrate material of a sensor.

Embodiment 39. The component of Embodiment 38, wherein the conductive permeable material comprises a plurality of conductive fibers.

Embodiment 40. The component of Embodiment 38, wherein the conductive permeable material comprises a metallic mesh.

FIG. 29 provides a cutaway view of exemplary devices according to the present disclosure. As shown in the left panel, device can include a porous and/or fibrous sensing region 3204 that is infused with a conductive material, e.g., MXene material, graphene, and the like. The sensing region 3204 can be disposed within an insulating material 3202, which material can be, e.g., PDMS, or other such material. The insulating material can be flexible, although this is not a requirement. A further insulating material 3206 (e.g., Parylene) can optionally be used to provide a further moisture barrier.

As shown in the middle panel, an aperture or other opening 3208 can be formed in the insulating material 3202 (and in the further insulating material 3206, if present) so as to expose the sensing region 3204. The aperture can be circular, but this is not a requirement. As shown, the top surface of the sensing region can be located beneath the top surface of the insulating material 3202 as well as beneath the top surface of the further insulating material 3206.

As shown in the right panel, a conductive pillar (or extension) 3208 can be provided; the pillar can be in electrical communication with sensing region 3204. (The conductive pillar can physically contact sensing region 3204.) As shown, conductive pillar 3208 can extend through aperture 3208.

Conductive pillar 3208 can be formed of the same material as sensing region 3204, but this is not a requirement. As shown, the conductive pillar can extend beyond the upper surface of insulating material 3204 and also beyond the upper surface of further insulating material 3206.

### REFERENCES

Heikenfeld, J. et al. Wearable sensors: Modalities, challenges, and prospects. Lab Chip 18, 217-248 (2018).
2. Rogers, J. A., Someya, T. & Huang, Y. Materials and mechanics for stretchable electronics. Science (80-.). 327, 1603-1607 (2010).
3. Liu, Y., Pharr, M. & Salvatore, G. A. Lab-on-Skin: A Review of Flexible and Stretchable Electronics for Wearable Health Monitoring. ACS Nano 11, 9614-9635 (2017).
4. Vitale, F. & Litt, B. Bioelectronics: the promise of leveraging the body's circuitry to treat disease. Bioelectron. Med. 1, 3-7 (2018).
5. Norton, J. J. S. et al. Soft, curved electrode systems capable of integration on the auricle as a persistent brain-computer interface. Proc. Natl. Acad. Sci. U.S.A. 112, 3920-3925 (2015).
6. Jeong, J. W. et al. Materials and optimized designs for human-machine interfaces via epidermal electronics. Adv. Mater. 25, 6839-6846 (2013).
7. Xu, B. et al. An Epidermal Stimulation and Sensing Platform for Sensorimotor Prosthetic Control, Management of Lower Back Exertion, and Electrical Muscle Activation. Adv. Mater. 28, 4462-4471 (2016).
8. Lee, S. et al. Toward Bioelectronic Medicine—Neuromodulation of Small Peripheral Nerves Using Flexible Neural Clip. Adv. Sci. 4, (2017).
9. DeLong, M. R. & Wichmann, T. Basal ganglia circuits as targets for neuromodulation in Parkinson disease. JAMA Neurology 72, 1354-1360 (2015).
10. Irisawa, H., Brown, H. F. & Giles, W. Cardiac pacemaking in the sinoatrial node. Physiological Reviews 73, 197-227 (1993).
11. Hwang, G. T. et al. Self-powered cardiac pacemaker enabled by flexible single crystalline PMN-PT piezoelectric energy harvester. Adv. Mater. 26, 4880-4887 (2014).
12. Ulloa, L., Quiroz-Gonzalez, S. & Torres-Rosas, R. Nerve Stimulation: Immunomodulation and Control of Inflammation. Trends in Molecular Medicine 23, 1103-1120 (2017).
13. Cheng, J. et al. Potential of Electrical Neuromodulation for Inflammatory Bowel Disease. Inflamm. Bowel Dis. 26, 1119-1130 (2020).
14. Granat, M. H., Ferguson, A. C. B., Andrews, B. J. & Delargy, M. The role of functional electrical stimulation in the rehabilitation of patients with incomplete spinal cord injury observed benefits during gait studies. Paraplegia 31, 207-215 (1993).
15. Peckham, P. H. & Knutson, J. S. Functional Electrical Stimulation for Neuromuscular Applications. Annu. Rev. Biomed. Eng. 7,327-360 (2005).
16. Ethier, C., Oby, E. R., Bauman, M. J. & Miller, L. E. Restoration of grasp following paralysis through brain-controlled stimulation of muscles. Nature 485, 368-371 (2012).
17. Nawrocki, R. A. et al. Self-Adhesive and Ultra-Conformable, Sub-300 nm Dry Thin-Film Electrodes for Surface Monitoring of Biopotentials. Adv. Funct. Mater. 28, 1-11 (2018).
18. Tian, L. et al. Large-area MRI-compatible epidermal electronic interfaces for prosthetic control and cognitive monitoring. Nat. Biomed. Eng. 3, 194-205 (2019).
19. Kim, D. H. et al. Epidermal electronics. Science (80-.). 333, 838-843 (2011).
20. Viventi, J. et al. Flexible, foldable, actively multiplexed, high-density electrode array for mapping brain activity in vivo. Nat. Neurosci. 14, 1599-605 (2011).
21. Khodagholy, D. et al. Highly conformable conducting polymer electrodes for in vivo recordings. Adv. Mater. (2011). doi:10.1002/adma.201102378
22. Li, J., Wang, P. & Huang, H. J. Dry Epidermal Electrodes Can Provide Long-Term High Fidelity Electromyography for Limited Dynamic Lower Limb Movements. Sensors (Basel). 20, (2020).
23. Searle, A. & Kirkup, L. A direct comparison of wet, dry and insulating bioelectric recording electrodes. Physiol. Meas. 21, 271-283 (2000).
24. Jensen, G. C., Krause, C. E., Sotzing, G. A. & Rusling, J. F. Inkjet-printed gold nanoparticle electrochemical arrays on plastic. Application to immunodetection of a cancer biomarker protein. Phys. Chem. Chem. Phys. 13, 4888-4894 (2011).
25. Lewinski, N., Colvin, V. & Drezek, R. Cytotoxicity of nanopartides. Small 4, 26-49 (2008).
26. Mattana, G. et al. Inkjet-Printing: A New Fabrication Technology for Organic Transistors. Advanced Materials Technologies 2, (2017).
27. Gao, M., Li, L. & Song, Y. Inkjet printing wearable electronic devices. Journal of Materials Chemistry C 5, 2971-2993 (2017).
28. Kraft, U., Molina-Lopez, F., Son, D., Bao, Z. & Murmann, B. Ink Development and Printing of Conducting Polymers for Intrinsically Stretchable Interconnects and Circuits. Adv. Electron. Mater. 6, (2020).
29. Park, S. et al. Hydrazine-reduction of graphite- and graphene oxide. Carbon N.Y. 49, 3019-3023 (2011).
30. Mattevi, C. et al. Evolution of electrical, chemical, and structural properties of transparent and conducting chemically derived graphene thin films. Adv. Funct. Mater. 19, 2577-2583 (2009).
31. Hantanasirisakul, K. et al. Fabrication of Ti₃C₂Tₓ MXene Transparent Thin Films with Tunable Optoelectronic Properties. Adv. Electron. Mater. 2, 1600050 (2016).
32. Mariano, M. et al. Solution-processed titanium carbide MXene films examined as highly transparent conductors †. 8, 16371 (2016).
33. Kim, S. J. et al. Interfacial assembly of ultrathin, functional mxene films. ACS Appl. Mater. Interfaces 11, 32320-32327 (2019).
34. Quain, E. et al. Direct Writing of Additive-Free MXene-in-Water Ink for Electronics and Energy Storage. Adv. Mater. Technol. 4, 1800256 (2019).
35. Zhang, C. et al. Additive-free MXene inks and direct printing of micro-supercapacitors. Nat. Commun. 10, 1795 (2019).
36. Vitale, F., Driscoll, N. & Murphy, B. Biomedical Applications of MXenes. in 2D Metal Carbides and Nitrides (MXenes) 503-524 (Springer International Publishing, 2019). doi:10.1007/978-3-030-19026-2_26
37. Han, X. et al. 2D Ultrathin MXene-Based Drug-Delivery Nanoplatform for Synergistic Photothermal Ablation and Chemotherapy of Cancer. Adv. Healthc. Mater. 7,1701394 (2018).
38. Li, L. et al. Hydrophobic and Stable MXene — Polymer Pressure Sensors for Wearable Electronics. Cite This ACS Appl. Mater. Interfaces 12, 15362-15369 (2020).
39. Seyedin, S. et al. MXene Composite and Coaxial Fibers with High Stretchability and Conductivity for Wearable Strain Sensing Textiles. Adv. Funct. Mater. 30, 1910504 (2020).
40. Driscoll, N. et al. Two-Dimensional Ti₃C₂ MXene for High-Resolution Neural Interfaces. ACS Nano 12, 10419-10429 (2018).
41. Driscoll, N. et al. Fabrication of Ti₃C₂ MXene Microelectrode Arrays for In Vivo Neural Recording. J. Vis. Exp. 15, 1-9 (2020).
42. Murphy, B. B. et al. A Gel-Free Ti3C2Tx-Based Electrode Array for High-Density, High-Resolution Surface Electromyography. Adv. Mater. Technol. 2000325, 1-10 (2020).
43. Zhang, C. J. et al. Oxidation Stability of Colloidal Two-Dimensional Titanium Carbides (MXenes). Chem. Mater. 29, 4848-4856 (2017).
44. Shuck, C. E. et al. Scalable Synthesis of Ti3C2Tx MXene. Adv. Eng. Mater. 22, 1901241 (2020).
45. Alhabeb, M. et al. Guidelines for Synthesis and Processing of Two-Dimensional Titanium Carbide (Ti3C2Tx MXene). Chem. Mater 29, 7633-7644 (2017).
46. Cogan, S. F., Troyk, P. R., Ehrlich, J., Plante, T. D. & Detlefsen, D. E. Potential-biased, asymmetric waveforms for charge-injection with activated iridium oxide (AIROF) neural stimulation electrodes. IEEE Trans. Biomed. Eng. 53, 327-332 (2006).
47. Grill, W. M. & Mortimer, J. T. Stimulus Waveforms for Selective Neural Stimulation. IEEE Eng. Med. Biol. Mag. 14, 375-385 (1995).
48. Cogan, S. F. Neural stimulation and recording electrodes. Annu. Rev. Biomed. Eng. 10, 275-309 (2008).
49. Ling, Z. et al. Flexible and conductive MXene films and nanocomposites with high capacitance. Proc. Natl. Acad. Sci. U.S.A. 111, 16676-81 (2014).
50. Zhang, C. J. et al. Transparent, Flexible, and Conductive 2D Titanium Carbide (MXene) Films with High Volumetric Capacitance. Adv. Mater. 29, 1702678 (2017).
51. Anasori, B., Lukatskaya, M. R. & Gogotsi, Y. 2D metal carbides and nitrides (MXenes) for energy storage. Nature Reviews Materials 2, (2017).
52. Ganji, M., Tanaka, A., Gilja, V., Halgren, E. & Dayeh, S. A. Scaling Effects on the Electrochemical Stimulation Performance of Au, Pt, and PEDOT:PSS Electrocorticography Arrays. Adv. Funct. Mater. 27, 1703019 (2017).
53. Hewson, D. J., Langeron, Y. & Duche, J. Evolution in impedance at the electrode-skin interface of two types of surface EMG electrodes during long-term recordings. J. Electromyogr. Kinesiol. 13, 273-279 (2003).
54. Li, G., Wang, S. & Duan, Y. Y. Towards gel-free electrodes: A systematic study of electrode-skin impedance. Sensors Actuators, B Chem. 241, 1244-1255 (2017).
55. Lee, S. M. et al. Self-adhesive epidermal carbon nanotube electronics for tether-free long-term continuous recording of biosignals. Sci. Rep. 4, (2014).
56. Chen, Y. H. et al. Soft, comfortable polymer dry electrodes for high quality ECG and EEG recording. Sensors (Switzerland) 14, 23758-23780 (2014).
57. Ferree, T. C., Luu, P., Russell, G. S. & Tucker, D. M. Scalp electrode impedance, infection risk, and EEG data quality. Clin. Neurophysiol. 112, 536-544 (2001).
58. Klimesch, W. EEG alpha and theta oscillations reflect cognitive and memory performance: A review and analysis. Brain Research Reviews 29, 169-195 (1999).
59. Hughes, S. W. & Crunelli, V. Thalamic mechanisms of EEG alpha rhythms and their pathological implications. Neuroscientist (2005). doi: 10.1177/1073858405277450
60. Li, Y. et al. An EEG-based BCI system for 2-D cursor control by combining Mu/Beta rhythm and P300 potential. IEEE Trans. Biomed. Eng. 57, 2495-2505 (2010).
61. Hwang, H.-J., Kwon, K. & Im, C.-H. Neurofeedback-based motor imagery training for brain-computer interface (BCI). J. Neurosci. Methods 179, 150-156 (2009).
62. Drost, G., Stegeman, D. F., van Engelen, B. G. M. & Zwarts, M. J. Clinical applications of high-density surface EMG: A systematic review. Journal of Electromyography and Kinesiology 16, 586-602 (2006).
63. Martinez-Valdes, E., Laine, C. M., Falla, D., Mayer, F. & Farina, D. High-density surface electromyography provides reliable estimates of motor unit behavior. Clin. Neurophysiol. 127, 2534-2541 (2016).
64. Chang, E. et al. A Review of Spasticity Treatments: Pharmacological and Interventional Approaches. Crit. Rev. Phys. Rehabil. Med. 25, 11-22 (2013).
65. Yelnik, A. P., Simon, 0., Parratte, B. & Gracies, J. M. How to clinically assess and treat muscle overactivity in spastic paresis. Journal of Rehabilitation Medicine 42, 801-807 (2010).
66. Masuda, T. & Sadoyama, T. Distribution of innervation zones in the human biceps brachii. J. Electromyogr. Kinesiol. 1, 107-115 (1991).
67. Morris, T. L. & Miller, J. C. Electrooculographic and performance indices of fatigue during simulated flight. in Biological Psychology 42, 343-360 (Elsevier B.V., 1996).
68. Usakli, A. B., Gurkan, S., Aloise, F., Vecchiato, G. & Babiloni, F. On the use of electrooculogram for efficient human computer interfaces. Comput. Intell. Neurosci. 2010, (2010).
69. Berman, J. I., Berger, M. S., Mukherjee, P. & Henry, R. G. Diffusion-tensor imaging—guided tracking of fibers of the pyramidal tract combined with intraoperative cortical stimulation mapping in patients with gliomas. (2004).
70. Morrell, M. J. Responsive cortical stimulation for the treatment of medically intractable partial epilepsy. Neurology 77, 1295-1304 (2011).
71. Lee, B. et al. Engineering Artificial Somatosensation Through Cortical Stimulation in Humans. Front. Syst. Neurosci. 12, 24 (2018).
72. Schenck, J. F. The role of magnetic susceptibility in magnetic resonance imaging: MRI magnetic compatibility of the first and second kinds. Med. Phys. 23, 815-850 (1996).
73. Duyn, J. H. The future of ultra-high field MRI and fMRI for study of the human brain. NeuroImage 62, 1241-1248 (2012).
74. Davis, K. A. et al. Glutamate imaging (GluCEST) lateralizes epileptic foci in nonlesional temporal lobe epilepsy. Sci. Transl. Med. 7, (2015).
75. Neumann, W., Pusch, T. P., Siegfarth, M., Schad, L. R. & Stallkamp, J. L. CT and MRI compatibility of flexible 3D-printed materials for soft actuators and robots used in image-guided interventions. Med. Phys. 46, 5488-5498 (2019).
76. Fan, Z. et al. Modified MXene/Holey Graphene Films for Advanced Supercapacitor Electrodes with Superior Energy Storage. Adv. Sci. 5, 1800750 (2018).
77. Daley, H., Englehart, K., Hargrove, L. & Kuruganti, U. High density electromyography data of normally limbed and transradial amputee subjects for multifunction prosthetic control. J. Electromyogr. Kinesiol. 22, 478-484 (2012).
78. Farina, D., Lorrain, T., Negro, F. & Jiang, N. High-density EMG E-Textile systems for the control of active prostheses. 32nd Annu. Int. Conf. IEEE Eng. Med. Biol. 3591-3593 (2010). doi:10.1109/IEMBS.2010.5627455
79. Cai, K. et al. Magnetic resonance imaging of glutamate. nature.com 3, 2 (2012).
80. Natu, V. et al. Edge Capping of 2D-MXene Sheets with Polyanionic Salts To Mitigate Oxidation in Aqueous Colloidal Suspensions. Angewandte Chemie—International Edition 58, 12655-12660 (2019).
81. Shahzad, F., Iqbal, A., Zaidi, S. A., Hwang, S. W. & Koo, C. M. Nafion-stabilized two-dimensional transition metal carbide (Ti3C2Tx MXene) as a high-performance electrochemical sensor for neurotransmitter. J. Ind. Eng. Chem. 79, 338-344 (2019).

82. Zhao, X. et al. Antioxidants Unlock Shelf-Stable Ti3C2Tx (MXene) Nanosheet Dispersions. Matter 1, 513-526 (2019).

83. Zhang, J. et al. Reduction of graphene oxide via 1-ascorbic acid. Chem. Commun. 46, 1112-1114 (2010).
84. Ganji, M., Tanaka, A., Gilja, V., Halgren, E. & Dayeh, S. A. Supp: Scaling Effects on the Electrochemical Stimulation Performance of Au, Pt, and PEDOT:PSS Electrocorticography Arrays. Adv. Funct. Mater. 27, (2017).
85. Geller, E.B., et al., Brain-responsive neurostimulation in patients with medically intractable mesial temporal lobe epilepsy. Epilepsia, 2017. 58(6): p. 994-1004.
86. Ung, H., et al., Intracranial EEG fluctuates over months after implanting electrodes in human brain. Journal of neural engineering, 2017. 14(5): p. 056011.
87. Mormann, F. and R.G. Andrzejak, Seizure prediction: making mileage on the long and winding road. Brain, 2016. 139(6): p. 1625-1627.
88. Thakor, N., Translating the brain-machine interface. Science translational medicine, 2013. 5(210): p. 210.
89. Rosin, B., et al., Closed-loop deep brain stimulation is superior in ameliorating parkinsonism. Neuron, 2011. 72(2): p. 370-384.
90. Lega, B., et al., Deep brain stimulation in the treatment of refractory epilepsy: update on current data and future directions. Neurobiology of disease, 2010. 38(3): p. 354-60.
91. Sellers, E.W., T.M. Vaughan, and J.R. Wolpaw, A brain-computer interface for long-term independent home use. Amyotrophic lateral sclerosis, 2010. 11(5): p. 449-455.
92. Ayton, L., et al., First-in-Human Trial of a Novel Suprachoroidal Retinal Prosthesis. PLoS One, 2014. 9(12): p. e115239.
93. Rachitskaya, A.V., et al., Intraoperative OCT Imaging of the Argus II Retinal Prosthesis System. Ophthalmic Surgery, Lasers and Imaging Retina, 2016. 47(11): p. 999-1003.
94. Clark, G.M. and R.J. Hallworth, A multiple-electrode array for a cochlear implant. The Journal of Laryngology & Otology, 1976. 90(07): p. 623-627.
95. Grayden, D.B. and G.M. Clark, Implant design and development. Cochlear implants: a practical guide (Cooper H, Craddock L), 2006: p. 1-20.
96. Wissel, K., et al., Platinum corrosion products from electrode contacts of human cochlear implants induce cell death in cell culture models. PloS one, 2018. 13(5): p. e0196649.
97. Witt, U., et al., Biodegradable polymeric materials—not the origin but the chemical structure determines biodegradability. Angewandte Chemie International Edition, 1999. 38(10): p. 1438-1442.
98. Anasori, B., et al., Two-dimensional, ordered, double transition metals carbides (MXenes). ACS Nano, 2015. 9(10): p. 9507-9516.
100.Niu, X., et al., Characterizing and patterning of PDMS-based conducting composites. Advanced Materials, 2007. 19(18): p. 2682-2686.
101. Cogan, S. F., T. Plante, and J. Ehrlich. Sputtered iridium oxide films (SIROFs) for low-impedance neural stimulation and recording electrodes. in The 26th Annual International Conference of the IEEE Engineering in Medicine and Biology Society. 2004. IEEE.
102. Wilks, S.J., et al., Poly (3, 4-ethylene dioxythiophene)(PEDOT) as a micro-neural interface material for electrostimulation. Frontiers in neuroengineering, 2009. 2: P. 7.
103. Credou et al, J. Mater. Chem. B, 2013,1, 3277-3286

1. A component, comprising:
(a) one or more sensors, a sensor comprising:
(i) a permeable substrate material having an upper surface, the permeable substrate optionally being non-conductive; and
(ii) an electrically conductive material,
the electrically conductive material disposed within and/or on the permeable substrate material so as render the permeable substrate material conductive; and
(b) an insulating material,
the insulating material having an upper surface and a thickness, and
the insulating material defining at least one aperture extending through the thickness of the insulating material, the at least one aperture being in register with a sensing location on the upper surface of the permeable substrate material of a sensor.

2. The component of claim 1, wherein the electrically conductive material comprises a MXene material, graphene, graphene oxide, graphite, carbon black, a metal, a conductive polymer, or any combination thereof.

3. The component of claim 1, wherein at least two of the one or more sensors do not physically contact one another.

4. The component of claim 1, wherein at least one sensor comprises a curved portion.

5. The component of claim 1, wherein at least some of the sensing locations of the one or more sensors define a periodic array that lies in a plane.

6. The component of claim 1, wherein the sensing locations of the one or more sensors define a circle that lies in a plane.

7. The component of claim 1, further comprising a conductive extension contacting and extending from the sensing location of a sensor through the aperture of the insulating material so as to extend beyond the upper surface of the insulating material.

8. The component of claim 7, wherein the conductive extension comprises the electrically conductive material.

9. The component of claim 1, wherein at least two of the one or more sensors are individually electronically addressable.

10. The component of claim 1, wherein a sensor is characterized as having a variable cross-sectional dimension.

11. The component of claim 1, wherein the insulating material comprises a polymer.

12. The component of claim 1, wherein the permeable substrate material comprises a woven textile.

13. The component of claim 1, wherein the permeable substrate material comprises a woven textile.

14. The component of claim 1, wherein the permeable substrate material comprises a non-woven textile.

15. The component of claim 1, wherein the permeable substrate material comprises a porous material.

16. The component of claim 1, further comprising a sealant conformally disposed on the insulating material.

17. The component of claim 1, further comprising an electrocatalytic element in electronic communication with the sensing location.

18. The component of claim 1, further comprising a biosensing element in electronic communication with the sensing location.

19. The component of claim 1, wherein the component is configured for implantation into a subject.

20. A method, comprising: collecting a signal with a component according to claim 1.

21. (canceled)

22. A device, the device comprising: one or more components according to claim 1.

23. The device of claim 22, wherein the device is characterized as being an electromyography (EMG) device, an electroencephalogram (EEG) device, electrocardiogram (EKG), a skin conductivity device, a body area network device, a strain sensor, a pressure sensor, a temperature sensor, a skin conductivity sensor, an electrostimulation device, an implantable sensing or stimulation device, a chemical sensor, or any combination thereof

24. A method, comprising:
infusing a fluid that comprises a carrier and a conductive material into a permeable substrate portion and then removing at least some of the carrier,
the infusing and removing being carried out under such conditions that the conductive material renders the permeable substrate conductive;
disposing an electrically insulating material over the permeable substrate, the electrically insulating material having an upper surface and defining a thickness;
optionally disposing a sealant over the electrically insulating material;
forming an opening through the thickness of the electrically insulating material, the opening being in register with a sensing location on the permeable substrate.

25. The method of claim 24, further comprising patterning the permeable substrate portion from a larger portion of the permeable substrate.

26. The method of claim 25, wherein the patterning comprises laser cutting, mechanical cutting, mechanical etching, chemical etching, or any combination thereof.

27. The method of claim 24, wherein the conductive material is characterized as being hydrophilic.

28. (canceled)

29. (canceled)

30. (canceled)

31. The method of claim 24, wherein the electrically insulating material comprises a polymer, the polymer optionally being elastomeric.

32. The method of claim 24, wherein the electrically insulating material comprises PDMS.

33. (canceled)

34. The method of claim 24, wherein the conductive material comprises MXene material, graphene, graphene oxide, graphite, carbon black, a metal, a conductive polymer, or any combination thereof.

35. The method of claim 24, wherein the permeable substrate portion comprises cellulose, polyester, or any combination thereof.

36. The method of claim 24, further comprising disposing a conductive extension that contacts and extends from the sensing location through the aperture of the insulating material so as to extend beyond the upper surface of the insulating material.

37. (canceled)

38. (canceled)

39. A component, comprising:
(a) one or more sensors, a sensor comprising:
a conductive permeable substrate material having an upper surface; and
(b) an insulating material,
the insulating material having an upper surface and a thickness, and
the insulating material defining at least one aperture extending through the thickness of the insulating material, the at least one aperture being in register with a sensing location on the upper surface of the permeable substrate material of a sensor.

40. The component of claim 39, wherein the conductive permeable material comprises a plurality of conductive fibers.

41. The component of claim 40, wherein the conductive permeable material comprises a metallic mesh.
